(19) United States
(12) Patent Application Publication
(10) Pub. No.: US 2026/0258461 A1
(43) Pub. Date: Sep. 3, 2026

(54) METHOD FOR PRODUCING AN AQUEOUS SOLUTION CONTAINING D-PSICOSE

(71) Applicant: ANNIKKI GMBH, Raaba-Grambach (AT)

(72) Inventors: Nicole STAUNIG, Raaba-Grambach (AT); Maria DUPONT, Raaba-Grambach (AT)

(21) Appl. No.: 19/164,835

(22) PCT Filed: Mar. 15, 2024
(86) PCT No.: PCT/EP2024/057013

§ 371 Date: Sep. 12, 2025

(30) Foreign Application Priority Data
Mar. 15, 2023 (EP) 23162092.3
May 15, 2023 (EP) 23173451.8
Dec. 22, 2023 (EP) 23219873.9
Dec. 22, 2023 (EP) 23219887.9

(51) Int. Cl. C12P 19/02

(52) U.S. Cl. CPC C12P 19/02

(57) ABSTRACT

The present invention relates to a method for preparing an aqueous solution containing D-psicose by forming a first D-psicose from a D-fructose, which is present in an aqueous solution, by treatment with an epimerase in vitro, after which the first D-psicose is reduced to allitol by treatment with a respective NAD(P)H-dependent oxidoreductase in vitro and, after deactivation and/or ultrafiltration of the epimerase, added a respective NAD(P)⁺-dependent oxidoreductase for forming D-psicose, after which the deactivated epimerase and the oxidoreductases are removed.

The present invention relates to a method for preparing an aqueous solution containing D-psicose.

### BACKGROUND OF THE INVENTION

### D-Psicose

The monosaccharide D-psicose, also known as D-allulose, is a ketohexose that is rarely found in nature (Zhang et al., 2016). It has been detected, among others, in the leaves of sweetspires (Itea sp.) (Hough & Stacey, 1966), but is also found in processed foods such as confectionery and spice sauces, where it is formed from D-fructose, its C₃ epimer, under the influence of heat (Oshima et al., 2006).

D-Psicose is interesting for the food industry because of its sweet taste. Compared to sucrose, D-psicose has a relative sweetening power of 70%, but a low energy content (0.2 kcal/g), which corresponds to a caloric reduction of approximately 95% (compared to sucrose) (Jiang et al., 2020).

In the US, D-psicose has been recognized by the US Food and Drug Administration (FDA) as a Generally Recognized as Safe (GRAS) sweetener, however, in the EU it has not been approved yet (Ahmed et al., 2022).

In addition, D-psicose has positive effects on lipid metabolism and carbohydrate metabolism (e.g., antidiabetic) and is anti-inflammatory and antioxidant (Zhang et al., 2016; Jiang et al., 2020; Chen et al., 2022).

Due to its scarcity in nature, D-psicose is mainly produced synthetically (chemically or biotechnologically).

The epimerization of D-fructose to D-psicose can be carried out by boiling in pyridine under reflux, followed by removal of the other hexoses by yeast fermentation, although only 6.8% of the theoretical yield of D-psicose is achieved (Doner, 1979). Another method involves the epimerization of D-fructose with molybdate ions as a catalyst, whereby only 0.5% of the D-fructose is converted to D-psicose (Bilik & Tihlarik, 1974). The inefficient chemical synthesis routes have in the meantime been replaced by more efficient biotechnological processes.

In 1993, Izumori et al. described a ketose-3-epimerase from Pseudomonas cichorii ST-24 for producing D-psicose from D-fructose (Izumori et al., 1993), which was also patented (EP 0592202 B1). Ketose-3-epimerases can be divided into three groups depending on their substrate specificity: 1) D-tagatose-3-epimerase (DTE), 2) D-psicose-3-epimerase (DPE) or D-allulose-3-epimerase (DAE), and 3) L-ribulose-3-epimerase (LRE) (Zhang et al., 2016; Jiang et al., 2020).

However, the conversion of D-fructose to D-psicose via ketose-3-epimerases does not proceed completely, but an equilibrium relationship between the two epimers is formed. Depending on the reaction conditions (temperature between 40 and 70° C., pH between 6 and 11), it is between 80:20 and 62.5:37.5 (D-fructose:D-psicose). Many of the epimerases also require a divalent metal ion such as Mn²⁺ or Co²⁺ (toxic) as co-factor (Zhang et al., 2016; Jiang et al., 2020).

The equilibrium during epimerization can be influenced not only by the temperature or pH value, but also by the addition of (toxic) borate. Due to the preferred formation of a D-psicose-borate complex, the equilibrium shifts toward D-psicose (Kim et al., 2008; Lim et al., 2009). EP 3643786 A2 and U.S. Ser. No. 11/028,420 B2 describe the chromatographic separation of the D-psicose-borate complex using simulated moving bed (SMB) chromatography. EP 3395952 B1 and U.S. Ser. No. 10/550,414 B2 disclose that the conversion during epimerization using DPE can be increased to up to 67% with the addition of sodium aluminate and to up to 52% with potassium iodate (comparison: 25% without the addition of aluminate or iodate).

Zhu et al. (2020) presented a system consisting of two enzymes (exo-inulase from Bacillus velezenis and DAE from Ruminococcus sp.) by means of which inulin from Helianthus tuberosus L. (Jerusalem artichoke) can be converted into a syrup consisting of D-glucose, D-fructose, and D-psicose (1:3:1). Li et al. (2021a) used a system consisting of invertase, D-glucose isomerase, and immobilized DAE from Pirellula sp. SH-Sr6A to convert sucrose, D-glucose, and D-fructose (from fruit juices) to D-psicose. They were able to enrich the juices with 16-19% of D-psicose (based on the total carbohydrate content).

In a study, Juneja et al. (2019) analyzed the techno-economic aspects of a modified corn dry grind process in which, in addition to ethanol, D-psicose is also produced from ground corn using a modified yeast strain (through expression of a DPE). The authors calculated that 390.4 l of ethanol and 75.3 kg of D-psicose can be obtained from one ton of corn and that the minimum selling price for D-psicose produced using the described process is 1.29 US$/kg (compared to the market price of 10-20 US$/kg in 2018). WO 2020/057560 A1 and WO 2020/057561 A1 describe the production of D-psicose from starch by saccharification, enzymatic isomerization, and epimerization.

Patel et al. (2018) used Smt3-DPE (fusion protein) immobilized on magnetic iron oxide nanoparticles to produce D-psicose from D-fructose from fruit pomace washing solutions. The immobilized epimerase was able to convert 20% of the D-fructose and was separated with a magnet after the reaction was completed.

Yang et al. (2018) transferred the DPE gene from Agrobacterium tumefaciens into the thermotolerant bacteria Kluyveromyces marxianus. Thus, 190 g/l of D-psicose could be produced from 750 g/l of D-fructose in 12 h at 55° C., with the remaining D-fructose being fermented to ethanol by K. marxianus. Dedania et al. (2020) immobilized DPE from A. tumefaciens on titanium dioxide nanoparticles and were able to convert 36% of the D-fructose to D-psicose. In addition, the immobilized enzyme could be reused up to nine times.

The D-fructose/D-psicose mixtures produced during the epimerization of D-fructose can be separated either by chromatographic methods or by the “biological method” (fermentation of the excess D-fructose to, e.g., ethanol) (Jiang et al., 2020). US 2021/0189441 A1 describes the separation of a D-fructose/D-psicose mixture, wherein the D-fructose is converted to L-lactic acid by a probiotic microorganism (Lactobacillus or Saccharomyces). EP 3423460 B1 describes a process for purifying a D-fructose/D-psicose mixture and obtaining high-purity D-psicose. EP 3553069 A1 and Van Duc Long et al. (2009) disclose a method for separating D-psicose and D-fructose based on SMB chromatography.

However, the production of D-psicose via the epimerase route has the following disadvantages: 1) position of the equilibrium on the D-fructose side, 2) addition of (partially toxic) metal ions as co-factors for many epimerases, 3) low activity and long-term stability of the epimerases, and 4) complex separation of the product mixture.

One option to circumvent thermodynamically unfavorable epimerization are enzyme cascades with phosphorylated intermediates. The final step, dephosphorylation, is irreversible and thus drives the cascade (Li et al., 2021b).

A cascade described in almost identical form by Li et al. (2021b) as well as in U.S. Ser. No. 11/168,342 B2 and U.S. Ser. No. 10/907,182 B2 shows D-glucose-1-phosphate (G1P) as the central intermediate. G1P is first converted to D-glucose-6-phosphate (G6P) by phosphoglucomutase and then further converted to D-fructose-6-phosphate (F6P) by glucose-6-phosphate isomerase. F6P is then epimerized by D-allulose-6-phosphate epimerase to D-psicose-6-phosphate, which is subsequently dephosphorylated by D-allulose-6-phosphate phosphatase to D-psicose.

G1P can be produced directly, for example, by the action of phosphorylases on, e.g., maltose and amylodextrins (obtained by the hydrolysis of starch), cellodextrins (obtained by the hydrolysis of cellulose), or sucrose, with the consumption of phosphate. Since the terminal sugar monomers of oligo- and polysaccharides cannot be phosphorylated by the corresponding phosphorylases, polyphosphate glucokinase (D-glucose 4 G6P) or polyphosphate fructokinase (D-fructose→F6P) have to be used, wherein polyphosphates must additionally be added as a phosphate source in order to increase the yields (U.S. Ser. No. 11/168,342 B2; U.S. Ser. No. 10/907,182 B2). The substrate used for the cascade by Li et al. (2021b) is starch, which is converted to D-psicose with yields of 79% (at a substrate concentration of 50 g/l; reaction time 24 h).

Wang et al. (2020) also developed an enzymatic cascade for the production of D-psicose starting from starch, which, however, is converted to glyceraldehyde-3-phosphate and dihydroxyacetone phosphate in several steps. Dihydroxyacetone phosphate is converted to D-psicose-1-phosphate with D-glyceraldehyde under the activity of L-fuculose-1-phosphate aldolase (FucA), which is dephosphorylated to D-psicose by phosphatase (95% yield at a titer of 15.2 mM). Glyceraldehyde-3-phosphate is further converted to 2-deoxy-D-ribose (Wang et al., 2020).

An enzyme cascade starting from glycerol is also described in the literature. It is converted to dihydroxyacetone phosphate (phosphorylation of glycerol with an acid phosphatase and subsequent oxidation with a glycerol phosphate oxidase) and D-glyceraldehyde (oxidation of glycerol with allitol oxidase), which in turn serve as substrates for an aldolase (such as FucA). After cleavage of the phosphate group, a mixture of D-sorbose and D-psicose is obtained, which can be separated chromatographically (Li et al., 2020). WO 2016/201110 A1, on the other hand, describes a method for producing D-psicose from dihydroxyacetone and D-glyceraldehyde using fructose-6-phosphate aldolase and DTE (intermediate product D-fructose).

Xiao et al. coupled the epimerization of D-fructose with the conversion of D-psicose to D-psicose-1-phosphate using L-rhamnulose kinase (consuming adenosine triphosphate (ATP)) to shift the epimerization equilibrium. Subsequent cleavage of the phosphate group by an acid phosphatase yields D-psicose (99% conversion of 20 mM of D-fructose). However, ATP must be regenerated with the addition of polyphosphate using a polyphosphate kinase (Xiao et al., 2019).

A major disadvantage of routes with phosphorylated intermediates is the use of expensive, energy-rich phosphate compounds such as polyphosphate or ATP in stoichiometric amounts to introduce the phosphate groups. By using phosphorylases, this problem can be partially avoided, but terminal monosaccharides cannot be phosphorylated without the aid of energy-rich phosphate compounds. In addition, the remaining phosphate compounds and phosphate ions must be removed after the reaction is complete.

Fermentation processes for the production of D-psicose are also known. Zhang et al. (2021) presented a fermentation process based on the co-cultivation of engineered Bacillus subtilis and Escherichia coli for the joint production of D-psicose (titer 11.7 g/l; conversion: 69.5%) and the lipase enzyme. US 2017/0298400 A1 describes the expression of DPE (e.g., from Agrobacterium tumefaciens) in various microorganisms. EP 3088515 B1 and U.S. Pat. No. 9,701,953 B2 describe a D-psicose-producing Ensifer adhaerens strain. EP 2470668 B1 discloses the immobilization of a GRAS microorganism (Corynebacterium glutamicum KCCM 11046) with expressed DPE on a sodium alginate carrier.

### Allitol as an Intermediate

The unfavorable position of the equilibrium of the epimerization of D-fructose to D-psicose can also be favorably influenced by downstream redox reactions. By combining a DTE with a ribitol dehydrogenase (RDH; EC 1.1.1.56) and formate dehydrogenase (FDH; as regeneration enzyme for the nicotinamide adenine dinucleotide NADH co-factor), D-Fructose can be converted in vitro to the allitol sugar alcohol (Takeshita et al., 2000).

Allitol can then be converted back to D-psicose via an oxidation step. This can be achieved, for example, microbially with Enterobacter aerogenes IK7 (complete oxidation of 100 g/l of allitol in 24 h) or Bacillus pallidus Y25 (48% conversion of 50 g/l of allitol in 48 h) (Gullapalli et al., 2007; Poonperm et al., 2007).

Due to its symmetry, the achiral allitol sugar alcohol forms an interface between the D- and L-hexoses in the so-called Izumoring strategy for the bioproduction of rare sugars (Izumori, 2006; Hassanin et al., 2017). It can therefore also serve as a precursor for the production of other rare monosaccharides.

The theoretical papers by Hold et al. (2009) and Siedentop et al. (2021) address the optimization of enzyme cascades. It is described that all components and a plurality of parameters must be taken into account, especially parameters regarding the cascade design, the enzymes themselves, the reaction conditions and environment, and also the process design, and that it cannot be predicted which ones will be successful. The synthesis of D-psicose is not specifically mentioned.

Chen et al. (2022) turn to the fermentation route via whole-cell biocatalysts (“in vivo”) for the production of D-psicose and conclude that this is the only route, with different optimizations, having the potential for the production of D-psicose economically and on an industrial scale in the future.

The advantages of whole-cell biocatalysts are obvious:
(1) cells containing enzymes in their interior are more easily accessible than the enzymes themselves, the purification of which is often laborious,
(2) the interior of the cells provides a suitable microenvironment for the enzymes and also allows co-factor regeneration (NAD(P)+/NAD(P)H),
(3) the cell walls and membranes protect the enzymes against the environment of the reaction medium, and
(4) the co-localization of multiple enzymes within the cells favors local enzyme concentrations and reduces the diffusion of intermediates in cascade reactions.

Consequently, the authors see “microbial cell factories” as the best opportunity to produce D-psicose on a large scale, so that normal consumers will also be able to enjoy this rare sugar in the near future.

A team led by Wang et al. (2022, 2023) is also working on the enzymatic biotransformation of sugars, investigating biotransformations in vitro and in vivo. Their goal is the development of an economical production method that can be carried out on an industrial scale.

For the production of D-psicose from D-fructose, Wang et al. (2023) describe an in vivo method consisting of two E. coli whole-cell biocatalysts, wherein in the first step (conversion of D-fructose to allitol), E. coli cells are used that include DPE from Clostridiales, an RDH from Providencia alcalifaciens, an FDH from Starkeya, as well as a further DPE from Rhizobium straminoryzae. In this manner, D-fructose (500 mM=90 g/l) was converted to 452 mM of allitol within 12 h at 37° C. and pH 6 using 1000 mM of sodium formate (two equivalents based on D-fructose) (conversion 90.4%). Approximately 30 mM of D-sorbitol were formed as by-product. The cells were separated by centrifugation and leaked proteins in the allitol-containing supernatant were deactivated by heat.

Then, E. coli cells containing an RDH from Rubrivivax sp. and an NADH oxidase from Streptococcus pyogenes were added to the allitol solution. Allitol (452 mM) turned into D-psicose (450 mM) within 24 h at pH 7.

Wang et al. (2023) further describe that, to their knowledge, the conversion rate of 90% is the highest conversion rate ever achieved for the production of D-psicose from D-fructose and announce their intention to further optimize the in vivo pathway, as the theoretical conversion rate of the proposed two-step process is 100%. A disadvantage of the process according to Wang et al. is the formation of the by-product D-sorbitol, which is formed by the reduction of D-fructose.

This is where the object of the present invention comes in, setting the goal of further improving the two-step process for forming D-psicose from D-fructose and providing a method for preparing aqueous solutions containing D-psicose, which can, in particular, also be carried out in a one-pot method.

### DETAILED DESCRIPTION OF THE INVENTION

According to the invention, this object is achieved by forming a first D-psicose from D-fructose, which is present in an aqueous solution, by treatment with an epimerase in vitro, after which the first D-psicose is reduced to allitol by treatment with a respective NAD(P)H-dependent oxidoreductase in vitro and, after deactivation and/or ultrafiltration of the epimerase, added a respective NAD(P)⁺-dependent oxidoreductase for forming D-psicose, after which the deactivated epimerase and the oxidoreductases are removed. Instead of deactivating the epimerase, it is also possible to immobilize it on or in a carrier material and filter it from the aqueous solution together with the carrier.

Surprisingly, it has been shown that practically no undesirable D-sorbitol is formed in the method according to the invention and that the yield of D-psicose can even be easily increased further towards 100%.

The process according to the invention is therefore not carried out fermentatively, but the enzymes are contained as such in the aqueous solution. According to the invention, the process is therefore carried out in vitro.

A preferred variant of the inventive method is characterized in that the NAD(P)⁺-dependent oxidoreductase for the formation of D-psicose from allitol comprises an amino acid sequence selected from the group consisting of:
i) an amino acid sequence having at least 80% identity to SEQ ID NO: 4, SEQ ID NO: 6, SEQ ID NO: 8, SEQ ID NO: 10, or SEQ ID NO: 12,
ii) an amino acid sequence encoded by a nucleic acid having at least 80% identity to SEQ ID NO: 3, SEQ ID NO: 5, SEQ ID NO: 7, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 5, SEQ ID NO: 7, SEQ ID NO: 9, or SEQ ID NO: 11, or a functional fragment thereof.

A “functional fragment” of this NAD(P)⁺-dependent oxidoreductase comprises an N-terminal and/or C-terminal truncated variant of the oxidoreductase with the amino acid sequence of SEQ ID NO: 4, SEQ ID NO: 6, SEQ ID NO: 8, SEQ ID NO: 10, or SEQ ID NO: 12, which has at least 50%, preferably at least 60%, more preferably at least 70%, even more preferably at least 80%, even more preferably at least 90%, and even more preferably at least 95% enzyme activity compared to the non-truncated oxidoreductase.

The method according to the invention is schematically shown in the accompanying FIGURE.

A preferred variant of the inventive method consists in that the oxidized co-factor NAD(P)⁺ formed by the reduction of D-psicose to allitol is reduced by means of an alcohol dehydrogenase (ADH) and a secondary alcohol with the formation of a ketone, wherein the secondary alcohol is preferably D-glucose or 2-propanol (isopropanol). 2-Propanol is a very inexpensive hydrogen donor for the regeneration of NAD(P)H, and the oxidation product acetone is easily separable due to its volatility (Xu et al., 2021). Acetone recovered from the exhaust gas stream can be heterogeneously-catalytically re-hydrogenated to 2-propanol (Al-Rabiah et al., 2022), either in the gas phase or in solution, present as 2-propanol/acetone/water mixtures or acetone/water mixtures, while hydrogen from sustainable sources (“green hydrogen”) could increasingly be used in the future.

The regeneration of the co-factor using ADH is, e.g., known from EP 2812439 B1 or was described in Xu et al. (2021).

In a further preferred embodiment of the present invention, the oxidized co-factor NAD(P)⁺ formed by the reduction is reduced by means of a glucose dehydrogenase and D-glucose with the formation of D-gluconate. The use of a glucose dehydrogenase for the regeneration of NAD(P)H is particularly advantageous because during the reduction of NAD(P)⁺, D-gluconate is formed from D-glucose, which can be obtained from the reaction mixture and used in multiple areas (e.g., in metal pickling agents, in medicines, and as stabilizers in food, etc.). In addition, the use of glucose dehydrogenase allows for the use of a mixture comprising D-fructose and D-glucose as a substrate for the production of allitol or D-psicose without adding additional D-glucose to the reaction mixture and without D-glucose being isomerized to D-fructose in advance. Mixtures of D-fructose and D-glucose can, for example, be produced by hydrolysis of sucrose. Particularly preferred, a glucose dehydrogenase originating from Priestia megaterium and comprising an amino acid sequence available under the NCBI accession number MDQ0804260.1 is used.

A further preferred variant of the method according to the invention comprises the use of a formate dehydrogenase (FDH) for the regeneration of the oxidized cofactor NAD(P)⁺ produced by the reduction by means of a formate dehydrogenase and formate (e.g., sodium formate) with the formation of CO₂.

A further preferred variant of the inventive method is characterized in that it is carried out as a one-pot reaction without isolation of any intermediate products.

In the inventive method, the enzymes are preferably used as a lysate of the corresponding cells producing them. In contrast to the method described by Wang et al. (2023), which is based on E. coli whole-cell biocatalysts with co-expressed recombinant enzymes, the enzymes are expressed individually in suitable E. coli production strains.

This allows for an optimization of the enzyme ratios and is therefore independent of the expression level in the overall construct compared to Wang et al. (2023).

Before executing the final step (oxidation), the enzymes (epimerase and reductase and/or dehydrogenase) of the first step (D-fructose→allitol) are deactivated by heat and/or removed by ultrafiltration in order to prevent the formation of by-products by the enzymes. Without appropriate treatment, a large part of the D-psicose formed by oxidation would be converted back to D-fructose.

The regeneration of the nicotinamide-based co-factors (NAD or NADP) occurs, in the case of a reduction of D-psicose to allitol, with an NAD(P)-dependent alcohol dehydrogenase, glucose dehydrogenase, or formate dehydrogenase, and in the case of the oxidation reactions (second step) with an H₂O-forming NAD(P)H oxidase.

The particularly preferred concentration of D-fructose is 50-250 g/l.

The particularly preferred temperature range for the first step (epimerization and reduction) is between 25 and 45° C., for the second step (oxidation) between 20 and 30° C.

The particularly preferred pH range of both steps is between 7 and 8.5.

In a further preferred embodiment of the inventive method, the enzymes are present in a suspension and/or in the homogenate and/or lysate of the respective cells forming them, with a lysate being particularly preferred.

In this context, suspension refers to a suspension of resting cells. These are harvested after cultivation (separated from the growth medium) und used as a paste or suspended in a suitable buffer system. In contrast to fermentative methods, where whole cells are also used, the resting cells cannot grow any longer because of the lack of carbon sources and nutrients, but only serve for converting substrates (Lin & Tao, 2017). In this context, homogenate refers to a physically and/or chemically treated suspension (e.g., by means of pressure, lysozyme, or ultrasound), so that the cell components are released from the cells. A lysate is obtained when insoluble cell components of the homogenate are removed, for example by filtration or centrifugation (see Production of the enzymes & preparation of the lysates for details).

In another variant, the enzymes can also be modified at the N-terminus with a water-soluble polymer such as polyethylene glycol, immobilized in or on a solid matrix, or be part of a fusion protein.

In another variant, the enzymes can be present in powder form, in lyophilized or spray-dried form.

After separation of the enzymes, D-psicose is most preferably present in an aqueous solution, from which solid D-psicose can, for example, be obtained by spray drying (US 2019/0315790 A1; Kawakami et al., 2013; Kawakami et al., 2014).

Due to the high purity of the obtained solution, it is possible to concentrate the filtrate and obtain the D-psicose in crystalline form or in the form of a syrup.

In a further preferred variant, the D-psicose is present in a syrup, wherein the syrup is prepared by concentrating the filtrate described above or by dissolving crystalline D-psicose, which can be prepared by the method according to the invention, in water. The syrup according to the invention preferably has a total solids content of approximately 50 wt. % to approximately 90 wt. %. The D-psicose content in the syrup according to the invention is approximately 80 wt. % to approximately 99 wt. % based on the dry substance.

Accordingly, a further aspect of the present invention relates to a syrup comprising D-psicose, which can be produced by the method according to the invention.

In a particularly preferred embodiment of the method, only enzymes from the enzyme groups of epimerases and oxidoreductases are used for the conversion of the starting material, with one or more of these enzymes being selected from each of these groups.

The epimerase used in the method can be from one of the groups EC 5.1.3.30 (D-psicose-3-epimerase) or EC 5.1.3.31 (D-tagatose-3-epimerase/L-ribulose-3-epimerase), the former being particularly preferred.

The enzymes used for the reduction of D-psicose and for the oxidation of allitol are from the group of oxidoreductases (see Table 1 for details).

The alcohol dehydrogenase (ADH) used for co-factor regeneration can belong to one of the EC 1.1.1.1 (NAD-dependent ADH) and EC 1.1.1.2 (NADP-dependent ADH) groups.

The NAD(P)-dependent alcohol dehydrogenase for co-factor regeneration preferably comprises or consists of an amino acid sequence selected from the group consisting of:
i) an amino acid sequence having at least 80% identity to SEQ ID NO: 18,
ii) an amino acid sequence encoded by a nucleic acid having at least 80% identity to SEQ ID NO: 17 and
iii) an amino acid sequence encoded by a nucleic acid that, under stringent conditions, binds to a nucleic acid molecule comprising the nucleic acid sequence of SEQ ID NO: 17.

Particularly suitable for co-factor regeneration in general is an alcohol dehydrogenase whose amino acid sequence has at least 80% identity to SEQ ID NO: 18, or that is encoded by a nucleic acid that has at least 80% identity to SEQ ID NO: 17, or that binds, under stringent conditions, to a nucleic acid molecule comprising the nucleic acid sequence of SEQ ID NO: 17, or a functional fragment of this alcohol dehydrogenase. A “functional fragment” of the alcohol dehydrogenase comprises an N-terminal and/or C-terminal truncated variant of alcohol dehydrogenase with the amino acid sequence of SEQ ID NO: 18, which has at least 50%, preferably at least 60%, more preferably at least 70%, even more preferably at least 80%, even more preferably at least 90%, and even more preferably at least 95% enzyme activity compared to the non-truncated alcohol dehydrogenase.

SEQ ID NO: 17:
ATGAAAGCTGCAGTTGTGGAACAATTTAAAAAGCCGTTACAAGTGAAAGA
AGTGGAAAAACCTAAGATCTCATACGGGGAAGTATTAGTGCGCATCAAAG
CGTGTGGGGTATGCCATACAGACTTGCATGCCGCACATGGCGACTGGCCT
GTAAAGCCTAAACTGCCTCTCATTCCTGGCCATGAAGGCGTCGGTGTAAT
TGAAGAAGTAGGTCCTGGGGTAACACATTTAAAAGTTGGAGATCGCGTAG
GTATCCCTTGGCTTTATTCGGCGTGCGGTCATTGTGACTATTGCTTAAGC
GGACAAGAAACATTATGCGAACGTCAACAAAACGCTGGCTATTCCGTCGA
TGGTGGTTATGCTGAATATTGCCGTGCTGCAGCCGATTATGTCGTAAAAA
TTCCTGATAACTTATCGTTTGAAGAAGCCGCTCCAATCTTTTGCGCTGGT
GTAACAACATATAAAGCGCTCAAAGTAACAGGCGCAAAACCAGGTGAATG
GGTAGCCATTTACGGTATCGGCGGGCTTGGACATGTCGCAGTCCAATACG
CAAAGGCGATGGGGTTAAACGTCGTTGCTGTCGATTTAGGTGATGAAAAA
CTTGAGCTTGCTAAACAACTTGGTGCAGATCTTGTCGTCAATCCGAAACA
TGATGATGCAGCACAATGGATAAAAGAAAAAGTGGGCGGTGTGCATGCGA
CTGTCGTCACAGCTGTTTCAAAAGCCGCGTTCGAATCAGCCTACAAATCC
ATTCGTCGCGGTGGTGCTTGCGTACTCGTCGGATTACCGCCGGAAGAAAT
ACCTATTCCAATTTTCGATACAGTATTAAATGGAGTAAAAATTATTGGTT
CTATCGTTGGTACGCGCAAAGACTTACAAGAGGCACTTCAATTTGCAGCA
GAAGGAAAAGTAAAAACAATTGTCGAAGTGCAACCGCTTGAAAACATTAA
CGACGTATTCGATCGTATGTTAAAAGGGCAAATTAACGGCCGCGTCGTGT
TAAAAGTAGATTAA
SEQ ID NO: 18:
MKAAVVEQFKKPLQVKEVEKPKISYGEVLVRIKACGVCHTDLHAAHGDWP
VKPKLPLIPGHEGVGVIEEVGPGVTHLKVGDRVGIPWLYSACGHCDYCLS
GQETLCERQQNAGYSVDGGYAEYCRAAADYVVKIPDNLSFEEAAPIFCAG
VTTYKALKVTGAKPGEWVAIYGIGGLGHVAVQYAKAMGLNVVAVDLGDEK
LELAKQLGADLVVNPKHDDAAQWIKEKVGGVHATVVTAVSKAAFESAYKS
IRRGGACVLVGLPPEEIPIPIFDTVLNGVKIIGSIVGTRKDLQEALQFAA
EGKVKTIVEVQPLENINDVFDRMLKGQINGRVVLKVD

The alcohol dehydrogenase for co-factor regeneration mentioned here preferably comprises an amino acid sequence which has at least 80% identity to SEQ ID NO: 18 more preferably 85%, even more preferably 90%, even more preferably 95%, even more preferably 98%, even more preferably 99%, in particular 100%. Particularly preferably, the alcohol dehydrogenase according to the invention for co-factor regeneration comprises or consists of the amino acid sequence of SEQ ID NO: 18.

Alternatively, the alcohol dehydrogenase for co-factor regeneration preferably comprises an amino acid sequence encoded by a nucleic acid having at least 80% identity to SEQ ID NO: 17, more preferably 85%, even more preferably 90%, even more preferably 95%, even more preferably 98%, even more preferably 99%, in particular 100%. Particularly preferably, the nucleic acid encoding the alcohol dehydrogenase for co-factor regeneration according to the invention comprises or consists of the nucleic acid sequence of SEQ ID NO: 17.

A further aspect of the present invention relates to the use of an alcohol dehydrogenase for co-factor regeneration, wherein the alcohol dehydrogenase comprises or consists of an amino acid sequence selected from the group consisting of:
i) an amino acid sequence having at least 80% identity to SEQ ID NO: 18,
ii) an amino acid sequence encoded by a nucleic acid having at least 80% identity to SEQ ID NO: 17, and
iii) an amino acid sequence encoded by a nucleic acid that, under stringent conditions, binds to a nucleic acid molecule comprising the nucleic acid sequence of SEQ ID NO: 17, or a functional fragment thereof.

The alcohol dehydrogenases disclosed here can be used for the regeneration of NAD(P)⁺ or NAD(P)H, i.e., for the reduction of NAD(P)⁺ or the oxidation von NAD(P)H, in a variety of enzymatic reactions.

Particularly preferred is the use of the inventive alcohol dehydrogenases in the co-factor regeneration of NAD(P)⁺, which is formed during reduction of D-psicose to allitol by means of an NAD(P)H-dependent oxidoreductase.

The NAD(P)⁺ cofactor produced during the reduction of D-psicose to allitol is reduced to NAD(P)H by means of formate and a formate dehydrogenase with the formation of CO₂ (cofactor regeneration).

The use of a formate dehydrogenase comprising or consisting of the amino acid sequence of SEQ ID NO: 2 or a functional fragment of this formate dehydrogenase is particularly preferred. The preferred formate dehydrogenase used is preferably encoded by the nucleic acid sequence of SEQ ID NO: 1. A “functional fragment” of the formate dehydrogenase comprises an N-terminal and/or C-terminal truncated variant of the formate dehydrogenase with the amino acid sequence of SEQ ID NO: 2, which has at least 50%, preferably at least 60%, more preferably at least 70%, even more preferably at least 80%, even more preferably at least 90%, and even more preferably at least 95% enzyme activity compared to the non-truncated formate dehydrogenase.

SEQ ID NO: 1:
ATGGCGAAAATACTTTGCGTTCTCTATGACGATCCGGTCGACGGCTACCC
GAAGACCTATGCGCGCGACGACCTGCCGAAGATCGACCACTATCCGGGCG
GGCAGACGCTGCCCACGCCCAAGGCGATCGACTTCACGCCGGGCGCGCTG
CTCGGCTCGGTCTCCGGCGAGCTCGGCCTGCGCAAATACCTGGAAGCCAA
CGGCCATACCTTCGTCGTCACCTCCGATAAGGACGGCCCGGATTCGGTGT
TCGAGAGGGAACTCGTCGACGCCGACGTGGTGATCTCGCAGCCCTTCTGG
CCGGCCTATCTGACGCCCGAGCGCATCGCCAAGGCGAAGAACCTGAAGCT
CGCGCTCACCGCCGGCATCGGCTCCGATCATGTCGATCTTCAGTCAGCTA
TCGACCGTGGCATCACTGTGGCCGAAGTCACATATTGCAACTCGATCAGC
GTCGCCGAGCACGTGGTGATGATGATCCTCGGCCTGGTACGAAACTACAT
TCCCTCGCATGACTGGGCGCGCAAGGGCGGCTGGAACATAGCCGACTGCG
TAGAGCACTCCTACGACCTCGAGGGCATGACCGTCGGCTCGGTGGCCGCC
GGCCGCATCGGCCTCGCCGTGCTGCGCCGCCTCGCGCCGTTCGACGTGAA
GCTGCACTATACCGACCGCCACCGTCTGCCAGAAGCGGTCGAGAAGGAGC
TGGGCCTCGTCTGGCACGATACCCGCGAGGACATGTACCCGCATTGCGAC
GTGGTCACGCTCAACGTGCCGCTGCACCCCGAAACCGAGCACATGATCAA
TGACGAGACGCTGAAGCTGTTCAAGCGCGGCGCCTATATCGTCAACACCG
CCCGCGGCAAGCTCGCCGACCGCGACGCCATCGTCCGCGCGATCGAGAGC
GGGCAGCTCGCGGGCTATGCCGGCGACGTGTGGTTCCCGCAGCCGGCTCC
GAAGGACCACCCCTGGCGCACCATGAAGTGGGAAGGCATGACGCCGCACA
TCTCCGGCACCTCGCTCTCTGCCCAGGCGCGCTACGCGGCGGGCACGCGC
GAGATCCTCGAATGCTTCTTCGAGGGCCGGCCGATCCGCGACGAGTACCT
GATCGTGCAGGGCGGCGCGCTCGCCGGCACCGGCGCGCATTCCTACTCGA
AGGGCAATGCGACCGGCGGTTCGGAAGAGGCCGCGAAGTTCAAGAAGGCT
GGCTGA
SEQ ID NO: 2:
MAKILCVLYDDPVDGYPKTYARDDLPKIDHYPGGQTLPTPKAIDFTPGAL
LGSVSGELGLRKYLEANGHTFVVTSDKDGPDSVFERELVDADVVISQPFW
PAYLTPERIAKAKNLKLALTAGIGSDHVDLQSAIDRGITVAEVTYCNSIS
VAEHVVMMILGLVRNYIPSHDWARKGGWNIADCVEHSYDLEGMTVGSVAA
GRIGLAVLRRLAPFDVKLHYTDRHRLPEAVEKELGLVWHDTREDMYPHCD
VVTLNVPLHPETEHMINDETLKLFKRGAYIVNTARGKLADRDAIVRAIES
GQLAGYAGDVWFPQPAPKDHPWRTMKWEGMTPHISGTSLSAQARYAAGTR
EILECFFEGRPIRDEYLIVQGGALAGTGAHSYSKGNATGGSEEAAKFKKA
G

According to a further preferred embodiment of the present invention, the formate dehydrogenase used for cofactor regeneration comprises an amino acid sequence selected from the group consisting of:
i) an amino acid sequence having at least 80% identity to SEQ ID NO: 2,
ii) an amino acid sequence encoded by a nucleic acid having at least 80% identity to SEQ ID NO: 1, and
iii) an amino acid sequence encoded by a nucleic acid that, under stringent conditions, binds to a nucleic acid molecule comprising the nucleic acid sequence of SEQ ID NO: 1, or a functional fragment thereof.

The formate dehydrogenase preferably comprises an amino acid sequence having at least 80% identity to SEQ ID NO: 2, more preferably 85%, even more preferably 90%, even more preferably 95%, even more preferably 98%, even more preferably 99%, in particular 100%.

Alternatively, the formate dehydrogenase preferably comprises an amino acid sequence encoded by a nucleic acid having at least 80% identity to SEQ ID NO: 1, more preferably 85%, even more preferably 90%, even more preferably 95%, even more preferably 98%, even more preferably 99%, in particular 100%.

Another aspect of the present invention relates to the use of a formate dehydrogenase for cofactor regeneration or a functional fragment thereof, wherein the formate dehydrogenase comprises or consists of an amino acid sequence selected from the group consisting of:
i) an amino acid sequence having at least 80% identity to SEQ ID NO: 2,
ii) an amino acid sequence encoded by a nucleic acid having at least 80% identity to SEQ ID NO: 1, and
iii) an amino acid sequence encoded by a nucleic acid that, under stringent conditions, binds to a nucleic acid molecule comprising the nucleic acid sequence of SEQ ID NO: 1, or a functional fragment thereof.

The glucose dehydrogenase (GDH) used for co-factor regeneration can be from one of the groups EC 1.1.1.47 (glucose-1-dehydrogenase), EC 1.1.1.118 (glucose-1-dehydrogenase (NAD⁺)), EC 1.1.1.119 (glucose-1-dehydrogenase (NADP⁺)), or EC 1.1.1.360 (glucose/galactose-1-dehydrogenase).

The NAD(P)-dependent glucose dehydrogenase for co-factor regeneration preferably comprises or consists of an amino acid sequence selected from the group consisting of:
i) an amino acid sequence having at least 80% identity to SEQ ID NO: 20,
ii) an amino acid sequence encoded by a nucleic acid having at least 80% identity to SEQ ID NO: 19, and
iii) an amino acid sequence encoded by a nucleic acid that, under stringent conditions, binds to a nucleic acid molecule comprising the nucleic acid sequence of SEQ ID NO: 19.

Particularly suitable for co-factor regeneration in general is a glucose dehydrogenase the amino acid sequence of which has at least 80% identity to SEQ ID NO: 20, or which is encoded by a nucleic acid having at least 80% identity to SEQ ID NO: 99, or that binds, under stringent conditions, to a nucleic acid molecule with the nucleic acid sequence of SEQ ID NO: 19, or a functional fragment of this glucose dehydrogenase. A “functional fragment” of the glucose dehydrogenase comprises an N-terminal and/or C-terminal truncated variant of glucose dehydrogenase with the amino acid sequence of SEQ ID NO: 18, which has at least 50%, preferably at least 60%, more preferably at least 70%, even more preferably at least 80%, even more preferably at least 90%, and even more preferably at least 95% enzyme activity compared to the non-truncated alcohol dehydrogenase.

SEQ ID NO: 19:
ATGTATACAGATTTAAAAGATAAAGTAGTTGTAATTACAGGTGGATCAAC
AGGTTTAGGACGCGCAATGGCTGTTCGTTTCGGTCAAGAAGAAGCAAAAG
TTGTTATTAACTATTACAACAATGAAGAAGAAGCTTTAGATGCGAAAAAA
GAAGTAGAAGAAGCAGGCGGACAAGCAATCATCGTTCAAGGCGACGTAAC
AAAAGAAGAAGATGTTGTAAACCTTGTTCAAACAGCTATTAAAGAATTCG
GTACATTAGACGTTATGATTAATAACGCTGGTGTTGAAAACCCAGTTCCT
TCTCATGAGTTATCTTTAGACAACTGGAATAAAGTTATTGATACAAACTT
AACAGGTGCATTCTTAGGAAGCCGTGAAGCAATCAAATATTTTGTTGAAA
ACGACATTAAAGGAAACGTTATTAACATGTCTAGTGTTCATGAAATGATT
CCTTGGCCATTATTTGTTCATTACGCAGCAAGTAAAGGCGGTATGAAACT
AATGACGGAAACATTGGCTCTTGAATATGCGCCAAAAGGTATCCGCGTAA
ATAACATTGGACCAGGTGCGATGAACACACCAATTAACGCAGAGAAATTT
GCAGATCCTGTACAACGTGCAGACGTAGAAAGCATGATTCCAATGGGTTA
CATCGGTAAACCAGAAGAAGTAGCAGCAGTTGCAGCATTCTTAGCATCAT
CACAAGCAAGCTATGTAACAGGTATTACATTATTTGCTGATGGTGGTATG
ACGAAATACCCTTCTTTCCAAGCAGGAAGAGGCTAA
SEQ ID NO: 20:
MYTDLKDKVVVITGGSTGLGRAMAVRFGQEEAKVVINYYNNEEEALDAKK
EVEEAGGQAIIVQGDVTKEEDVVNLVQTAIKEFGTLDVMINNAGVENPVP
SHELSLDNWNKVIDTNLTGAFLGSREAIKYFVENDIKGNVINMSSVHEMI
PWPLFVHYAASKGGMKLMTETLALEYAPKGIRVNNIGPGAMNTPINAEKF
ADPVQRADVESMIPMGYIGKPEEVAAVAAFLASSQASYVTGITLFADGGM
TKYPSFQAGRG

The glucose dehydrogenase for co-factor regeneration mentioned here preferably comprises an amino acid sequence having at least 80% identity to SEQ ID NO: 20, more preferably 85%, even more preferably 90%, even more preferably 95%, even more preferably 98%, even more preferably 99%, in particular 100%. Particularly preferably, the inventive glucose dehydrogenase for co-factor regeneration comprises or consists of the amino acid sequence of SEQ ID NO: 20.

Alternatively, the glucose dehydrogenase for co-factor regeneration preferably comprises an amino acid sequence encoded by a nucleic acid having at least 80% identity to SEQ ID NO: 19, more preferably 85%, even more preferably 90%, even more preferably 95%, even more preferably 98%, even more preferably 99%, in particular 100%. Particularly preferably, the nucleic acid encoding the inventive glucose dehydrogenase for co-factor regeneration comprises or consists of the nucleic acid sequence of SEQ ID NO: 19.

A further aspect of the present invention relates to the use of a glucose dehydrogenase for co-factor regeneration, wherein the glucose dehydrogenase comprises or consists of an amino acid sequence selected from the group consisting of:
i) an amino acid sequence having at least 80% identity to SEQ ID NO: 20,
ii) an amino acid sequence encoded by a nucleic acid having at least 80% identity to SEQ ID NO: 19, and
iii) an amino acid sequence encoded by a nucleic acid that, under stringent conditions, binds to a nucleic acid molecule comprising the nucleic acid sequence of SEQ ID NO: 19, or a functional fragment thereof.

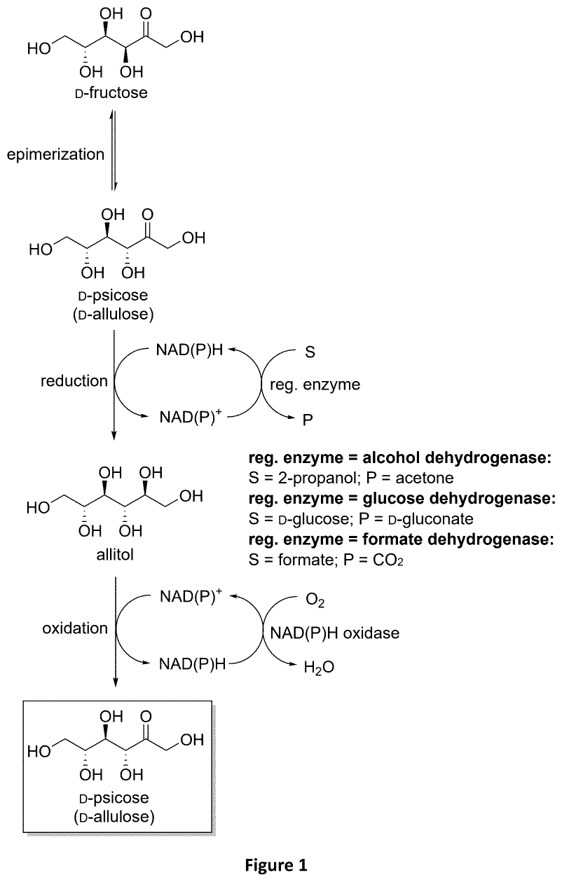

The NAD(P)H oxidase used for co-factor regeneration (see FIG. 1) can be from one of the groups of EC 1.6.3.1 (NAD(P)H oxidase (H₂O₂-forming)), EC 1.6.3.2 (NAD(P)H oxidase (H₂O-forming)), EC 1.6.3.3 (NADH oxidase (H₂O₂-forming)), and EC 1.6.3.4 (NADH oxidase (H₂O-forming)), wherein the H₂O-forming classes are particularly preferred.

A particularly preferred H₂O-forming NAD(P)H oxidase preferably comprises or consists of an amino acid sequence selected from the group consisting of:
i) an amino acid sequence having at least 80% identity to SEQ ID NO: 14 or SEQ ID NO: 16,
ii) an amino acid sequence encoded by a nucleic acid having at least 80% identity to SEQ ID NO: 13 or SEQ ID NO: 15, and
iii) an amino acid sequence encoded by a nucleic acid that, under stringent conditions, binds to a nucleic acid molecule comprising the nucleic acid sequence of SEQ ID NO: 13 or SEQ ID NO: 15, or
a functional fragment thereof.

A “functional fragment” of this NAD(P)H oxidase comprises an N-terminal and/or C-terminal truncated variant of the NAD(P)H oxidase with the amino acid sequence of SEQ ID NO: 4, SEQ ID NO: 6, SEQ ID NO: 8, SEQ ID NO: 10, or SEQ ID NO: 12, which has at least 50%, preferably at least 60%, more preferably at least 70%, even more preferably at least 80%, even more preferably at least 90%, and even more preferably at least 95% enzyme activity compared to the non-truncated NAD(P)H oxidase.

SEQ ID NO: 13:
ATGAGCAAAATTGTTATCGTGGGTGCAAATCATGCAGGCACCGCAGCAAT
TAATACCATTCTGGATAATTATGGCAGCGAAAATGAAGTGGTTGTGTTTG
ATCAGAATAGCAACATTAGCTTTCTGGGTTGTGGTATGGCACTGTGGATT
GGTAAACAAATTAGCGGTCCGCAGGGTCTGTTTTATGCAGATAAAGAAAG
CCTGGAAGCAAAAGGTGCCAAAATCTATATGGAAAGTCCGGTTACCGCCA
TTGATTATGATGCAAAACGTGTTACCGCACTGGTTAATGGTCAAGAACAT
GTTGAAAGCTACGAGAAACTGATTCTGGCAACCGGTAGCACCCCGATTCT
GCCTCCGATTAAAGGTGCAGCCATTAAAGAAGGTAGTCGCGATTTTGAAG
CAACCCTGAAAAATCTGCAGTTCGTGAAACTGTATCAGAATGCCGAAGAT
GTGATTAACAAACTGCAGGATAAAAGCCAGAATCTGAATCGTATTGCAGT
TGTTGGTGCAGGTTATATTGGTGTTGAACTGGCAGAAGCATTTAAACGTC
TGGGTAAAGAAGTGATTCTGATTGACGTTGTTGATACCTGTCTGGCAGGT
TATTATGATCAGGATCTGAGCGAAATGATGCGTCAGAATCTGGAAGATCA
TGGTATCGAACTGGCATTTGGTGAAACCGTTAAAGCAATTGAAGGTGATG
GTAAAGTGGAACGTATTGTTACCGATAAAGCAAGCCATGATGTGGATATG
GTTATTCTGGCAGTTGGTTTTCGTCCGAATACAGCACTGGGTAATGCAAA
ACTGAAAACCTTTCGTAATGGTGCCTTTCTGGTGGATAAAAAACAAGAAA
CCAGCATCCCGGATGTTTATGCAATTGGTGATTGTGCAACCGTGTATGAT
AATGCCATTAACGACACCAACTATATTGCACTGGCAAGCAATGCACTGCG
TAGCGGTATTGTTGCAGGTCATAATGCAGCCGGTCATAAACTGGAAAGTC
TGGGTGTTCAGGGTAGCAATGGTATTTCAATTTTTGGCCTGAATATGGTT
AGCACCGGTCTGACCCAAGAAAAAGCCAAACGTTTTGGTTATAATCCGGA
AGTTACCGCCTTTACCGATTTTCAGAAAGCCAGCTTTATCGAGCATGATA
ACTATCCGGTTACGCTGAAAATTGTGTATGACAAAGATAGCCGTCTGGTT
CTGGGTGCACAGATGGCCAGCAAAGAAGATATGAGCATGGGTATTCACAT
GTTTAGCCTGGCCATTCAAGAGAAAGTTACCATTGAACGTCTGGCCCTGC
TGGATTATTTCTTTCTGCCGCATTTTAATCAGCCGTACAACTATATGACC
AAAGCAGCACTGAAAGCCAAATAA
SEQ ID NO: 14:
MSKIVIVGANHAGTAAINTILDNYGSENEVVVFDQNSNISFLGCGMALWI
GKQISGPQGLFYADKESLEAKGAKIYMESPVTAIDYDAKRVTALVNGQEH
VESYEKLILATGSTPILPPIKGAAIKEGSRDFEATLKNLQFVKLYQNAED
VINKLQDKSQNLNRIAVVGAGYIGVELAEAFKRLGKEVILIDVVDTCLAG
YYDQDLSEMMRQNLEDHGIELAFGETVKAIEGDGKVERIVTDKASHDVDM
VILAVGFRPNTALGNAKLKTFRNGAFLVDKKQETSIPDVYAIGDCATVYD
NAINDTNYIALASNALRSGIVAGHNAAGHKLESLGVQGSNGISIFGLNMV
STGLTQEKAKRFGYNPEVTAFTDFQKASFIEHDNYPVTLKIVYDKDSRLV
LGAQMASKEDMSMGIHMFSLAIQEKVTIERLALLDYFFLPHFNQPYNYMT
KAALKAK
SEQ ID NO: 15:
ATGAAAGTAGTAGTAGTAGGCTGTACACATGCAGGAACAGCGGCAGTTAA
GACGATTTTAAATGAACATCCAGATGCATCAGTATCAGTATATGAGCGTA
ATGACAATGTCTCATTTCTATCTTGTGGGATTGCGTTGTATGTTGGTGGA
GTTGTGAAAGATCCTGCAGGTTTGTITTATTCAAGTCCAGAAGAACTTGC
ATCAATGGGCGCGAAAATTAACATGGAACACAATGTGAAAAATATAGATA
ATGAGAATAAGGTCGTAGTAATTGAGAATTTAAAAACAGGCGAAACATTT
GAAGAAAGCTATGATAAGTTGGTAATGACAACTGGATCATGGCCAATTAT
TCCTCCAATTGATGGAATCAATAGTGAAAATATTCTTTTGTGTAAAAACT
ATAACCAAGCAAATGAAATTATTAAAGAATCAAAAAATGCTAAAAAGATT
GTCATTGTTGGTGGTGGCTATATTGGAATTGAATTAGTTGAGGCATTTGC
AGAATCTGGCAAGCAAGTGACGCTAGTTGATGGATTAGATCGTATTTTAA
ACAAATATTTAGATGCTGAATTCACTTCTGTTTTAGAGCATGATTTACAA
GAAAGAGGCGTTACGCTAGCTTTAAACCAAACCGTCGAGAAATTTGTTGC
CAATGAATCAGGTGCTGTGACAGCTGTGAAAACACCAGTTGGAGAATATG
AGGCTGATTTAGTTATTTTATGTGTTGGATTTAAACCAAATACTGATTTG
TTGAAGGATAAAGTAGAGATGTTGCCAAATGGTGCCATCGTAGTGGATGA
ATATATGAGAACAAGCGATGAAGCGATTTTTGCTGCTGGCGATAGTTGCG
CGGTTCATTATAATCCAACTGGAGGCTCTGCGTATATTCCGTTAGCTACA
AATGCAGTTAGAATGGGAGCTTTAGTTGGGAAAAATATTGTTTCTCCAAC
AGTTAAATATCGTGGCACGCAAGCAACTTCTGGTTTATATTTATTTGGTT
TTAATATAGGTTCAACCGGATTGACTGAAAATAGCGCTCCTCATTTTGGC
GTAGAGGTTCGTTCAGTAGTTGTAGAAGATAATTATCGTCCAGAGTTTAT
GCCGACAACAGAGAAAGTAACGATGAAATTAGTTTATGAAGTAGGAACGA
ATCGGATTGTTGGAGGTCAAATCATGTCAAAATATGATGTGACACAATCT
GCCAATACGTTATCTTTATGTGTTCAAAATAAAATGACGATTGAGGATTT
GGCTTATGTAGATTTCTTCTTCCAACCTCACTTTGATCGTCCTTGGAACT
ATTTAAATATTTTAGCGCAAGCAGCTGTTGAGCAAGAGCGTAAACTAGCA
AAATAA
SEQ ID NO: 16:
MKVVVVGCTHAGTAAVKTILNEHPDASVSVYERNDNVSFLSCGIALYVGG
VVKDPAGLFYSSPEELASMGAKINMEHNVKNIDNENKVVVIENLKTGETF
EESYDKLVMTTGSWPIIPPIDGINSENILLCKNYNQANEIIKESKNAKKI
VIVGGGYIGIELVEAFAESGKQVTLVDGLDRILNKYLDAEFTSVLEHDLQ
ERGVTLALNQTVEKFVANESGAVTAVKTPVGEYEADLVILCVGFKPNTDL
LKDKVEMLPNGAIVVDEYMRTSDEAIFAAGDSCAVHYNPTGGSAYIPLAT
NAVRMGALVGKNIVSPTVKYRGTQATSGLYLFGFNIGSTGLTENSAPHFG
VEVRSVVVEDNYRPEFMPTTEKVTMKLVYEVGTNRIVGGQIMSKYDVTQS
ANTLSLCVQNKMTIEDLAYVDFFFQPHFDRPWNYLNILAQAAVEQERKLA
K

The preferably used H₂O-forming NAD(P)H oxidase preferably comprises or consists of an amino acid sequence having at least 80% identity to SEQ ID NO: 16 or SEQ ID NO: 14, more preferably 85%, even more preferably 90%, even more preferably 95%, even more preferably 98%, even more preferably 99%, in particular 100%. Particularly preferably, the H₂O-forming NAD(P)H oxidase comprises or consists of the amino acid sequence of SEQ ID NO: 16 or SEQ ID NO: 14.

Alternatively, the H₂O-forming NAD(P)H oxidase preferably has an amino acid sequence encoded by a nucleic acid having at least 80% identity to SEQ ID NO: 15 or SEQ ID NO: 13, more preferably 85%, even more preferably 90%, even more preferably 95%, even more preferably 98%, even more preferably 99%, in particular 100%. Particularly preferably, the nucleic acid encoding the H₂O-forming NAD(P)H oxidase comprises or consists of the nucleic acid sequence of SEQ ID NO: 15 or SEQ ID NO: 13.

A further aspect of the present invention relates to the use of an H₂O-forming NAD(P)H oxidase for co-factor regeneration (NAD(P)H to NAD(P)⁺) comprising or consisting of an amino acid sequence selected from the group consisting of:
i) an amino acid sequence having at least 80% identity to SEQ ID NO: 16 or SEQ ID NO: 14,
ii) an amino acid sequence encoded by a nucleic acid having at least 80% identity to SEQ ID NO: 15 or SEQ ID NO: 13, and
iii) an amino acid sequence encoded by a nucleic acid that, under stringent conditions, binds to a nucleic acid molecule comprising the nucleic acid sequence of SEQ ID NO: 15 or SEQ ID NO: 13, or
a functional fragment thereof.

In combination with co-factor regeneration, the enzymatic strategy presented here allows for a biocatalytic, environmentally friendly, and highly efficient production method for producing D-psicose.

NAD(P)H-dependent oxidoreductase for reducing the first D-psicose to allitol preferably comprises or consists of an amino acid sequence selected from the group consisting of:
i) an amino acid sequence having at least 80% identity to SEQ ID NO: 4, SEQ ID NO: 10, or SEQ ID NO: 12,
ii) an amino acid sequence encoded by a nucleic acid having at least 80% identity to SEQ ID NO: 3, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 9, or SEQ ID NO: 11.

Particularly suitable for reducing the first D-psicose to allitol or D-psicose to allitol in general is an oxidoreductase, the amino acid sequence of which has at least 80% identity to SEQ ID NO: 4, SEQ ID NO: 10, or SEQ ID NO: 12, or which is encoded by a nucleic acid having at least 80% identity to SEQ ID NO: 3, SEQ ID NO: 9, or SEQ ID NO: 1, or which, under stringent conditions, binds to a nucleic acid molecule comprising the nucleic acid sequence of SEQ ID NO: 3, SEQ ID NO: 9, or SEQ ID NO: 11. In addition, this oxidoreductase can surprisingly be used to oxidize allitol to D-psicose.

The NAD(P)⁺-dependent oxidoreductase for forming allitol from D-psicose preferably comprises or consists of an amino acid sequence selected from the group consisting of:
i) an amino acid sequence having at least 80% identity to SEQ ID NO: 4, SEQ ID NO: 6, SEQ ID NO: 8, SEQ ID NO: 10, or SEQ ID NO: 12,
ii) an amino acid sequence encoded by a nucleic acid having at least 80% identity to SEQ ID NO: 3, SEQ ID NO: 5, SEQ ID NO: 7, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 5, SEQ ID NO: 7, SEQ ID NO: 9, or SEQ ID NO: 11.

SEQ ID NO: 3:
ATGACACAGTCTCTTCAGGGCAAGATCGTCGCCATTACTGGCGCGGCTTC
GGGCATTGGCCTCGAATGCGCCCGCTATCTCATCGAAGCTGGCGCGGTGG
TCTATCTTCTGGACCGTGACGCCAAAACTCTCGAAGACAAAACGGCAGAA
CTCGGCAGCCAGGCCCATGCGATCATCGTCGATCTCTTCGACTACAAAAC
CGTAGATGCTGCGGTCGCGCAGATCGTTGAGGAGCAGGGCCGGATCGATG
TTTTCCACGCCAACGCCGGCGCGTATGTGGGCGGCAATGTCTGGGAAGGG
GATCCCGATAGCTGGGATGCGATGCTGCACCTGAACATCAATGCAGCTTT
CCGCTCGGTCCGTGCCGTGCTGCCGCAGATGATGAAGCAGGAAAGTGGCG
ATATCGTCATGACCAGCTCGATCGCGGGCATGATCCCCATCATGGCCGAG
CCGATCTACACGGCCTCGAAACATGCCGTGCAGGCATTCGTACATACGGT
GCGCCGACAGGTCGCGAAGTATGGAATTCGTGTGGGTGCAATCCAGCCTG
GTCCTGTAGTCACGCCTTTGCTGAAAGACTGGGATCAGGCCCGTCTTGAA
GCCAACATCAAAGCGGGTGCCCTGATGGAAGCCAAGGAAGTCGCCGAAGC
CCTGATCTTCATCCTGACACGTTCAAAGGGTGTCATGGTGCGGGATCTGG
TCGTTCTGCCACATAACTTCGACGCCTAAGCTTAAGCGGCCGCACTCGAG
CACCACCACCACCACCACTGAGATCCGGCTGCTAA
SEQ ID NO: 4:
MTQSLQGKIVAITGAASGIGLECARYLIEAGAVVYLLDRDAKTLEDKTAE
LGSQAHAIIVDLFDYKTVDAAVAQIVEEQGRIDVFHANAGAYVGGNVWEG
DPDSWDAMLHLNINAAFRSVRAVLPQMMKQESGDIVMTSSIAGMIPIMAE
PIYTASKHAVQAFVHTVRRQVAKYGIRVGAIQPGPVVTPLLKDWDQARLE
ANIKAGALMEAKEVAEALIFILTRSKGVMVRDLVVLPHNFDA
SEQ ID NO: 5:
ATGAGCACACCGGAAAATCTGAGCTTTGTGCTGCAGAAACCGTTTGATGT
GAAATTTGAAGATCGTCCGATTCCGAAACTGAGCGATCCGTATAGCGTTA
AAATTCAGGTGAAAAAAACCGGCATTTGCGGTAGTGATGTTCACTATTTC
ACCCATGGTGCAATTGGTGATTTTGTTGTTAAAGCACCGATGGTTCTGGG
TCATGAAAGCAGCGGTGTTGTTCTGGAAGTTGGTAGCGAAGTTAAAAGCC
TGAAAGTTGGTGATCGTGTTGCAATGGAACCGGGTGTTCCGAGCCGTCAT
AGTGATGAGTATAAAAGCGGTCGTTATAATCTGTGTCCGCACATGGCATT
TGCAGCAACCCCTCCGTATGATGGCACCCTGTGTAAATACTATATTCTGC
CGGAAGATTTCTGCGTTAAACTGCCGGAACATGTTAGCCTGGAAGAAGGT
GCACTGGTTGAACCGCTGAGCGTTGCAGTTCATAGCAGCAAACTGGGTAA
CATTAAACCGGGTAGCCATGTTGCAATTTATGGTGCAGGTCCGGTTGGTC
TGCTGGTTGCAGCAGTTGCAAGCGCATTTGGTGCAGAAAGCGTTACCATT
ATTGATCTGGTTGAAAGCCGTCTGAATCTGGCAAAAGAACTGGGTGCAAC
CGCAACCGTTCAGGTTGATTTTAAAGATACCCCGAAAGAAAGCGCAGCAA
AAGTTGTTGCAGCAAATAATGGCATTGCACCGGATGTTGTTATTGATGCA
AGCGGTGCAGAAGCAAGCATTAATTCAGCCATTAATGCAATTCGTCCGGG
TGGCACCTATGTTCAGGTGGGTATGGGTAAACCGGATGTGAGCTTTCCGA
TTGCAACCCTGATTGGTAAAGAACTGACCGTTAAAGGTAGCTTTCGTTAT
GGTTATGGTGATTATCCGCTGGCAGTTAGCCTGCTGGCAAGCGGTAAAGT
TAATGTGAAAAAACTGATCACCCATGAAGTGAAATTCGAGGATGCAGCAG
AAGCATTTCAGCTGGTTCGTGATGGTAAAGCCATTAAATGTATTATCAAC
GGTCCGGAATAA
SEQ ID NO: 6:
MSTPENLSFVLQKPFDVKFEDRPIPKLSDPYSVKIQVKKTGICGSDVHYF
THGAIGDFVVKAPMVLGHESSGVVLEVGSEVKSLKVGDRVAMEPGVPSRH
SDEYKSGRYNLCPHMAFAATPPYDGTLCKYYILPEDFCVKLPEHVSLEEG
ALVEPLSVAVHSSKLGNIKPGSHVAIYGAGPVGLLVAAVASAFGAESVTI
IDLVESRLNLAKELGATATVQVDFKDTPKESAAKVVAANNGIAPDVVIDA
SGAEASINSAINAIRPGGTYVQVGMGKPDVSFPIATLIGKELTVKGSFRY
GYGDYPLAVSLLASGKVNVKKLITHEVKFEDAAEAFQLVRDGKAIKCIIN
GPE
SEQ ID NO: 7:
ATGAATAACAACCTGCCGAAAACCATGAAAGCAGCAGTTATGCATGGCAC
CCGTGAAATTAGCATTGAAACCCTGCCGATTCCGCAGATTGATGAAAATG
AAGTTCTGATCAAAGTTATGGCCGTTGGTATTTGTGGTAGCGATCTGCAC
TATTACACCCAGGGTCGTATTGGTAAATACAAAGTGGAAAAACCGTTTAT
CCTGGGTCATGAATGTAGCGGTGAAGTTGTTGCAATTGGTAGCGCAGTTG
AACGTTTTCGTGTTGGTGATCGTGTTGCCGTTGAACCGGGTGTTACCTGT
GGTCATTGTGAAGCATGTAAAGAGGGTCGTTATAATCTGTGTCCGGATGT
TCAGTTTCTGGCAACCCCTCCGGTTGATGGTGCATTTGTTCAGTATATCA
AAATGCGCCAGGATTTCGTTTTTCTGATTCCGAATAGCCTGAGCTATGAA
GATGCAGCACTGATTGAACCGTTTAGCGTGGGTATTCATGCAGCAACCCG
TACCAAACTGCAGCCTGGTAGCACCATTGCAATTATGGGTATGGGTCCGG
TTGGTCTGATGGCAGTTGCAGCAGCAAAAGCATTTGGTGCAAGCACCATT
ATTGCAACCGATCTGGAACCGCTGCGTCTGGAAGCAGCCAAACGTATGGG
TGCAACCCATGTTATTAACATTCGTGAACAGGATCCGCTGAACGAGATTA
AAAACATTACCGAAAATGTGGGTGTTGATGTTGCATGGGAAACCGCAGGT
AATCCGAAAGCACTGCAGAGCAGCCTGAGCAGCATTCGTCGTGGTGGTAA
ACTGGCAATTGTTGGTCTGCCGAGCCAGAGCGATATTCCGCTGGATGTTC
CGTTTATTGCCGATAATGAAATCGATATCTATGGCATCTTTCGCTATGCA
AACACCTATCCGAAAGGCATCAAATTTCTGACCAGCGGTGCAATTGATAC
CAAAAATCTGGTTACCGATCGTTATCCGCTGGCAGGTACACGTGAAGCAA
TGGAACGTGCACTGAATTTCAAAAACGAATGCCTGAAAATCATCGTGTAT
CCGAACGAATAA
SEQ ID NO: 8:
MNNNLPKTMKAAVMHGTREISIETLPIPQIDENEVLIKVMAVGICGSDLH
YYTQGRIGKYKVEKPFILGHECSGEVVAIGSAVERFRVGDRVAVEPGVTC
GHCEACKEGRYNLCPDVQFLATPPVDGAFVQYIKMRQDFVFLIPNSLSYE
DAALIEPFSVGIHAATRTKLQPGSTIAIMGMGPVGLMAVAAAKAFGASTI
IATDLEPLRLEAAKRMGATHVINIREQDPLNEIKNITENVGVDVAWETAG
NPKALQSSLSSIRRGGKLAIVGLPSQSDIPLDVPFIADNEIDIYGIFRYA
NTYPKGIKFLTSGAIDTKNLVTDRYPLAGTREAMERALNFKNECLKIIVY
PNE
SEQ ID NO: 9:
ATGACCTCTCCTCTCCAGGGTAAGATAGCCGCCATCACGGGCGGGGCTTC
GGGCATCGGCCTCGAATGTGTCCGCCAGATCGCCGCAAGTGGTGCCACGG
TTTATATTCTCGACCGCGACCATCAGGCGCTCGACAAGGCGCGCGAAGAA
TTGGGCGAGCGCGTTCATACCATCGAGGTCGATCTCTTCCGTTACGAAAC
GGTCGATCGCGCCATCGAAACCATCGTGTCCGAACAAGGACGCATCGACA
TTCTCCATGTCAATGCGGGCGCGTATATCGGCGGCAATGTCTGGGAAGGC
GATCCCGATAAATGGGACAAGATGCTGAATCTCAACATCAACGCCGCCTT
CCGTTCCGCCCGCGCCGTCATGCCCGCCATGATGAAGCAGAAAAGCGGCG
ATATCATCATGACAAGCTCGATCGCAGGCATCGTCCCGATCCCGGCGGAG
CCGATCTACACGGCTTCCAAACATGCGGTGCAAGCCTTCGCTCACACCAT
ACGCCGCCAGTTGGCCCCGTTCGGCATCCGCGTCGGCGCCATCCAGCCCG
GCCCGGTCGTCACGCCCTTGCTCAATGATTGGGACCCCGAGCGCCTTAAA
GCCAATATCGAGGCTGGCGCCATGATGCAGCCTTCTGACGTCGCCGAAGC
CGTGGTTTTCATGCTGTCCCGCCGCAAGGGAACGGTAATCCGCGACTTGG
TTCTGTTACCCCATTCTTTCGACGTCTAA
SEQ ID NO: 10:
MTSPLQGKIAAITGGASGIGLECVRQIAASGATVYILDRDHQALDKAREE
LGERVHTIEVDLFRYETVDRAIETIVSEQGRIDILHVNAGAYIGGNVWEG
DPDKWDKMLNLNINAAFRSARAVMPAMMKQKSGDIIMTSSIAGIVPIPAE
PIYTASKHAVQAFAHTIRRQLAPFGIRVGAIQPGPVVTPLLNDWDPERLK
ANIEAGAMMQPSDVAEAVVFMLSRRKGTVIRDLVLLPHSFDV
SEQ ID NO: 11:
ATGGCTATATCTCTCGAAAACAACGTAGCTGCAATTACAGGTGCCGCTTC
AGGTATCGGTCTCGAATGTGCACGCACACTGATCAAAGCAGGCGCTAAAG
TTGTCCTCATTGACCGAGCAGAAGATAGACTAAATCAATTGGTCGCAGAA
TTAGGTGAAAATGCAATTCCATTAGTTATCGATTTAATGAAACCAGAACA
AGTCGATGGCATGTTAGCGCGTATTATCGAAAAGGCAGGCAGATTAGATA
TCTTTCATGCTAATGCTGGAGCTTACATTGGTGGGCCCGTAGCCGAAGGC
GATCCCGATGTTTGGGATAAAGTCCTAAATTTAAATGTTAATGCCGCATT
CCGCTGTGTTCGCGCAGTTCTACCACACTTTATCGCACAAAAGTCAGGCG
ATATTCTATTCACCAGCTCTATCGCTGGTATGGTTCCCGTAATTTGGGAG
CCTATTTACACGGCATCAAAATTTGCGGTTCAAGCATTCGTTCATTCTAC
TCGCCGTCAGGTTTCTGAACACGGTGTCCGTGTTGGTGCTGTATTACCTG
GTCCTGTTGTTACTGCGTTATTAGATGATTGGCCAAAAGAAAAACTTGAA
GAAGCTTTAGCTAACGGTAGTTTAATGCAACCCATTGAAGTTGCTGAGGC
TGTTCTATTCATGCTGACGCGTCCAAGAAATATTACAATTCGCGATTTAG
TTATTTTACCCAATAGTGTTGACCTCTAA
SEQ ID NO: 12:
MAISLENNVAAITGAASGIGLECARTLIKAGAKVVLIDRAEDRLNQLVAE
LGENAIPLVIDLMKPEQVDGMLARIIEKAGRLDIFHANAGAYIGGPVAEG
DPDVWDKVLNLNVNAAFRCVRAVLPHFIAQKSGDILFTSSIAGMVPVIWE
PIYTASKFAVQAFVHSTRRQVSEHGVRVGAVLPGPVVTALLDDWPKEKLE
EALANGSLMQPIEVAEAVLFMLTRPRNITIRDLVILPNSVDL

The oxidoreductases mentioned here for the reduction of D-psicose to allitol and/or for the oxidation of allitol to D-psicose preferably comprise an amino acid sequence having at least 80% identity to SEQ ID NO: 4, SEQ ID NO: 6, SEQ ID NO: 8, SEQ ID NO: 10, or SEQ ID NO: 12, more preferably 85%, even more preferably 90%, even more preferably 95%, even more preferably 98%, even more preferably 99%, in particular 100%. Particularly preferably the inventive oxidoreductase for the reduction of D-psicose to allitol and/or for the oxidation of allitol to D-psicose comprises or consists of the amino acid sequence of SEQ ID NO: 4, SEQ ID NO: 10, or SEQ ID NO: 12, and the oxidoreductase for the oxidation of allitol to D-psicose comprises or consists of one of the amino acid sequence of SEQ ID NO: 6 or SEQ ID NO: 8.

Alternatively, the oxidoreductases for the reduction of D-psicose to allitol and/or for the oxidation of allitol to D-psicose preferably comprise an amino acid sequence encoded by a nucleic acid having at least 80% identity to SEQ ID NO: 3, SEQ ID NO: 4, SEQ ID NO: 9, or SEQ ID NO: 11, more preferably 85%, even more preferably 90%, even more preferably 95%, even more preferably 98%, even more preferably 99%, in particular 100%. Particularly preferably, the nucleic acid encoding the inventive oxidoreductase for the reduction of D-psicose to allitol and/or the oxidation of allitol to D-psicose comprises or consists of the nucleic acid sequence of SEQ ID NO: 3, SEQ ID NO: 9, or SEQ ID NO: 11, and the oxidoreductase for the oxidation of allitol to D-psicose comprises or consists of the nucleic acid sequence of SEQ ID NO: 5 or SEQ ID NO: 7.

The term “identity” as used here refers to the percentage of identical nucleotides or amino acids between at least two nucleotide or amino acid sequences aligned using a standardized algorithm (“alignment”). Such an algorithm can, in a standardized and reproducible manner, insert gaps into the compared sequences to optimize the alignment between two sequences and thus achieve a more meaningful comparison of the two sequences.

The percentage identity between sequences can be determined using one or more computer algorithms or programs known in the state of the art or described herein. According to the invention, the Basic Local Alignment Search Tool (BLAST) (Altschul et al., 1990) provided by the National Center for Biotechnology Information (NCBI) is used to determine identity. The BLAST software suite includes several programs, including a tool called “BLAST 2 Sequences” which is used for the direct pairwise comparison of two nucleotide or amino acid sequences. “BLAST 2 Sequences” can also be interactively retrieved on the Internet via the NCBI World Wide Web page and used. The blastn program (for nucleotide sequences) uses as defaults a word length (W) of 11, an expectation (E) of 10, M=5, N=−4 and a comparison of both strands. For amino acid sequences, the blastp program uses as defaults a word length of 3 and an expectation (E) of 10 and the BLOSUM62 scoring matrix (Henikoff & Henikoff, 1989), alignments (B) of 50, expectation (E) of 10, M=5, N=−4.

Alternatively, the oxidoreductases for the reduction of D-psicose to allitol and/or the oxidation of allitol to D-psicose preferably comprise an amino acid sequence encoded by a nucleic acid that, under stringent conditions, binds to a nucleic acid molecule comprising the nucleic acid sequence of SEQ ID NO: 3, SEQ ID NO: 5, SEQ ID NO: 7, SEQ ID NO: 9, or SEQ ID NO: 11. As used herein, stringent conditions refer to conditions under which so-called specific hybrids, but no non-specific hybrids are formed. For example, stringent conditions include hybridization in 6×SSC (sodium chloride/sodium citrate) at 45° C. and then washing with 0.2 to 1×SSC, 0.1% SDS at 50 to 65° C.; or such conditions may include hybridization in 1×SSC at 65 to 70° C. and then washing with 0.3×SSC at 65 to 70° C.

Hybridization can be performed by conventionally known methods, such as those described by J. Sambrook et al. in Molecular Cloning, A Laboratory Manual, 2nd Ed., Cold Spring Harbor Laboratory (1989).

One aspect of the present invention relates to the use of an oxidoreductase for the reduction of D-psicose to allitol and/or the oxidation of allitol to D-psicose, wherein the oxidoreductase comprises an amino acid sequence selected from the group consisting of:
i) an amino acid sequence having at least 80% identity to SEQ ID NO: 4, SEQ ID NO: 10, or SEQ ID NO: 12,
ii) an amino acid sequence encoded by a nucleic acid having at least 80% identity to SEQ ID NO: 3, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 9, or SEQ ID NO: 11.

A further aspect of the present invention relates to the use of an oxidoreductase for the oxidation of allitol to D-psicose, wherein the oxidoreductase comprises an amino acid sequence selected from the group consisting of:
i) an amino acid sequence having at least 80% identity to SEQ ID NO: 4, SEQ ID NO: 6, SEQ ID NO: 8, SEQ ID NO: 10, or SEQ ID NO: 12,
ii) an amino acid sequence encoded by a nucleic acid having at least 80% identity to SEQ ID NO: 3, SEQ ID NO: 5, SEQ ID NO: 7, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 5, SEQ ID NO: 7, SEQ ID NO: 9, or SEQ ID NO: 11.

Depending on the reaction (reduction or oxidation), the inventive oxidoreductases require corresponding co-factors, as mentioned above.

### Materials

D-Psicose was purchased from TCI and Hunan Garden Naturals Inc. (China), allitol was purchased from TCI, D-fructose, NADPH tetrasodium salt, and methanol were purchased from PanReac AppliChem (ITW Reagents), D-glucose, sodium gluconate, IPTG (isopropyl-@-D-thiogalactopyranoside) were purchased from Sigma-Aldrich, potassium dihydrogene phosphate, di-potassium hydrogen phosphate, NAD⁺, NADH disodium salt, NADP⁺ disodium salt, and sodium dodecyl sulfate (SDS) were purchased from Carl Roth, and triethanolamine (TEA) was purchased from Chem-Lab NV.

### Production of the Enzymes & Preparation of the Lysates

General Information on the Expression of Recombinant Enzymes in E. coli

For recombinant enzyme production in an Escherichia coli strain, the gene to be expressed was first amplified in a PCR using the genomic DNA or its synthetic equivalent adapted to the codon usage of E. coli as a template together with specific oligonucleotides, which additionally carried recognition sequences for restriction endonucleases, and isolated from the reaction mixture. After nucleic acid digestion with the restriction enzymes SphI and HindIII, the gene fragment encoding the target enzyme was ligated into the SphI and HindIII-cleaved backbone of the expression vector pQE70-Kan.

The ligation product was transformed into chemically competent Top10F E. coli cells and the resulting colonies were used for plasmid isolation and restriction analysis.

The result of the cloning step was verified by restriction enzyme digestion and DNA sequencing. The resulting construct carries the target gene under the IPTG-inducible T5 promoter.

For overexpression of the enzyme in E. coli, the resulting expression plasmid was transformed into the competent expression cells RB791. After 24 h of incubation at 37° C., the resulting colonies were inoculated into LB medium for expression assays.

The next day, expression cultures with an optical density OD₅₅₀ of 0.02 were inoculated and shaken at 37° C. until an OD₅₅₀ of 0.3 was reached. Subsequently, the temperature was lowered to 25° C., and the cultures were induced with 0.1 mM of IPTG when an OD₅₅₀ of 0.5 was reached. After 22 h, the cultures were harvested (separated from the medium by centrifugation in the form of a cell pellet) and analyzed for the expression of the recombinant enzyme using SDS gel electrophoresis and an activity determination (use test or optical enzymatic assay).

### Preparation of Cell Lysates Using Sonifier Disruption

To prepare a cell suspension, the cell pellet prepared according to the above method was weighed into a suitable container and mixed with buffer and lysozyme (final concentration 0.5 mg/ml) (e.g. triethanolamine (TEA)-HCl) and dissolved with stirring. The mass fraction of biomass is usually 20%, the rest is the buffer.

A Branson Sonifier 450 was used for cell disruption. The suspension was treated three times with 15 ultrasonic pulses each (device settings: Timer=15; Duty Cycle=50; Output Control=3-5).

The resulting homogenate was centrifuged for 10 min at 4° C. and 16000 rpm (Eppendorf Centrifuge 5417R) to separate the insoluble cell fragments and obtain the lysate.

TABLE 1
Enzyme classes and donor organisms for the enzymes used in the examples
(SDR = oxidoreductase from the short-chain dehydrogenase/reductase family).

| Enzyme type | Catalyzed | | |
|---|---|---|---|
| (EC class) | reaction | Donor organism | Literature/SEQ ID NO: |
| D-Psicose-3-epimerase | D-fructose → | Clostridium | (Mu et al., 2011; Chan et al., |
| (EC 5.1.3.30) | D-psicose | cellulolyticum H10 | 2012) |
| SDR I | D-psicose ↔ | Gluconobacter frateurii | (NCBI Protein Database: |
| | allitol | (DSM 7146) | WP_063903495.1); |
| | | | SEQ ID NO: 4 |
| Xylitol dehydrogenase | allitol → | Galactocandida | (Habenicht et al., 1999); |
| (XDH; EC 1.1.1.9) | D-psicose | mastotermitis | SEQ ID NO: 6 |
| | | (Candida sp. HA167) | |
| NAD(P)-dependent | allitol → | Priestia megaterium | (NCBI Protein Database: |
| alcohol dehydrogenase | D-psicose | | WP_013084280.1); |
| | | | SEQ ID NO: 8 |
| SDR II | allitol ↔ | Kozakia baliensis | (NCBI Protein Database: |
| | D-psicose | | WP_070401870.1); |
| | | | SEQ ID NO: 10 |
| SDR III | allitol ↔ | Providencia heimbachae | (NCBI Protein Database: |
| | D-psicose | | WP_068907433.1); |
| | | | SEQ ID NO: 12 |
| Alcohol dehydrogenase | 2-propanol → | (Geo-)Bacillus | (Sakoda & Imanaka, 1992); |
| (ADH; EC 1.1.1.1) | acetone | stearothermophilus | SEQ ID NO: 18 |
| | | NCA1503 | |
| NADH oxidase I | NADH → NAD⁺ | Streptococcus mutans | (Matsumoto et al., 1996); |
| (EC 1.6.3.4) | | | SEQ ID NO: 14 |
| NADH oxidase II | NADH → NAD⁺ | Carnobacterium | SEQ ID NO: 16 |
| (EC 1.6.3.4) | | divergens | |
| Glucose dehydrogenase | D-glucose → | Priestia megaterium | (NCBI Protein Database: |
| (GDH; EC 1.1.1.47) | D-gluconate (via | | MDQ0804260.1); |
| | D-gluconolactone) | | SEQ ID NO: 20 |
| Formate dehydrogenase | formate → CO₂ | Starkeya novella/ | (NCBI Protein Database: |
| (EC 1.17.1.9) | | Ancylobacter novellus | WP_013168047.1); |
| | | | SEQ ID NO: 2 |

### Analytical Methods

### High Performance Liquid Chromatography

An Agilent HPLC 1260 Infinity II Series system was used to quantify D-psicose, D-fructose, D-glucose and allitol by HPLC (high performance liquid chromatography). Detection was carried out using a refractive index detector (RI detection). For the measurement, a Phenomenex Rezex RPM-Monosaccharide Pb+2 (8%) column with an appropriate pre-column was used and eluted isocratically with ultrapure water.

### High Performance Anion Exchange Chromatography

A Dionex ICS6000 system with AS-AP autosampler was used to quantify D-gluconic acid/D-gluconate using HPAEC (High Performance Anion Exchange Chromatography). The measurement was performed using conductivity detection (CD) coupled to a Dionex AERS 500 electrolytically regenerated suppressor in external water mode. A Dionex IonPac AS11-HC-4 μm column with an appropriate pre-column and an NaOH gradient was used to separate the analytes. The mobile phase was additionally pretreated with a Dionex ATC Anion Trap Column.

### Determination of Enzyme Activities (Optical-Enzymatic Assay)

Enzyme activities in the lysates were determined using a Shimadzu UV-1900 spectrophotometer. For this purpose, the formation or consumption of NAD(P)H was monitored at a wavelength of 340 nm via the change in absorption. The measurements were performed with 0.2 mM of co-factor (NAD(P)⁺ or NAD(P)H). For this purpose, 20 μl of a 10 mM stock solution of the co-factor were placed in a cuvette (Greiner Bio-One Semi-Micro Cuvette made of polystyrene), and the desired pH was adjusted with 100 mM of TEA HCl buffer (870 μl). 10 μl of lysate (diluted or undiluted) and 100 μl of substrate solution were added to the cuvette, and the measurement was started immediately. The measurements were by default carried out at 25° C. The enzyme activity of the lysate can be determined in U/ml (based on the volume of the lysate) or U/g (based on the biomass used for production) using the extinction coefficient of NADH/NADPH at 340 nm (E=6220 L mol⁻¹ cm⁻¹). Here, 1 U stands for 1 pmol substrate conversion per minute (1 U=1 pmol/min=1 μmol/min=1.67·10⁻⁸ kat).

The following examples describe preferred variants of the method according to the invention in more detail. The lysates used in these examples were prepared according to the procedures described above.

### Example 1

Production of D-Psicose from D-Fructose—Cofactor Regeneration with ADH and 2-Propanol

The reaction was carried out in a Labfors 5 tabletop bioreactor (Infors AG). A glass reactor (volume 3.4 l) with an agitator and a pH electrode was used as the vessel. The pH was controlled by adding 1M NaOH or 1M H₂SO₄.

At the start, 50 ml of a D-fructose solution (500 g/l), 246.1 ml of deionized water, and 96 ml of a 200 mM TEA-HCl buffer (pH 8) were placed in the reactor and brought to 35° C. with stirring.

To start the reaction, 25 ml of D-psicose 3-epimerase lysate was added. Then, 25 ml of SDR I lysate, 4 kU of alcohol dehydrogenase lysate, 10 ml of a 10 mM NAD⁺ solution, and 40 ml of 2-propanol were added.

During operation, samples were continuously taken from the reactor solution and analyzed as follows: 100 μl of the reactor solution was mixed with 200 μl of methanol and incubated in an Eppendorf Thermomixer at 60° C. and 1200 rpm for 15 min. The sample was briefly centrifuged in a centrifuge, mixed with 700 μl of deionized water, vortexed, and then centrifuged for 5 min at max. g. 200 μl of the supernatant was transferred to an HPLC vial with an insert and measured by HPLC (RI detection).

After 23 h of running time, 15 ml of 2-propanol was added, after 50 h, 20 ml was added, and after 77 h, 15 ml was added.

After 74 h, 5 ml of D-psicose 3-epimerase lysate was added, and after 77 h, 15 ml of SDR I lysate and 2.4 kU of alcohol dehydrogenase lysate was added.

After 97 h, 94% of the D-fructose (50 g/l) had been converted to allitol.

The entire reactor contents were then heated to 70° C. for 60 min (deactivation of D-psicose 3-epimerase) and, after cooling to 24° C., 25 ml of xylitol dehydrogenase lysate, 10 kU of NADH oxidase lysate, and 10 ml of a 10 mM NAD⁺ solution were added.

Allitol was completely oxidized to D-psicose within 3 h. The reactor contents were heated to 70° C., the pH was adjusted to 4, and the mixture was stirred for 30 minutes at 70° C. The enzymes were filtered using a glass frit (P3).

In this way, 94% of the D-fructose was converted to D-psicose. D-sorbitol could not be detected.

The filtrate was concentrated to a syrup with a D-psicose concentration of 520 g/l in a rotary evaporator, with any remaining acetone and 2-propanol also being separated off.

Example 1 shows that the epimerase can be denatured by heat (in a one-pot process) and that the resulting precipitate does not interfere with the further reaction.

### Example 2

Production of D-Psicose from D-Fructose—Cofactor Regeneration with GDH and D-Glucose

The reaction was carried out in a Multifors tabletop bioreactor (Infors AG). A glass reactor (volume 1 l) with an agitator and a pH electrode was used as vessel. The pH was controlled by adding 5M of NaOH or 1M of H₂SO₄.

At the start, 17.5 g of D-fructose and 17.5 g of D-glucose (final concentration 50 g/l each), 217.8 ml of deionized water, and 26.5 ml of a 500 mM potassium phosphate buffer (pH 7.5) were placed in the reactor and brought to 35° C. with stirring.

To start the reaction, 17.5 ml of D-psicose-3-epimerase lysate was added. Then 24.5 ml of SDR I lysate, 0.4 kU of glucose dehydrogenase lysate, and 3.5 ml of a 10 mM NAD+ solution were added.

During operation, samples were continuously taken from the reactor solution and analyzed as follows: 100 μl of the reactor solution was mixed with 200 μl of methanol and incubated in an Eppendorf Thermomixer at 60° C. and 1200 rpm for 15 min. The sample was briefly centrifuged, mixed with 700 μl of deionized water, vortexed, and then centrifuged for 5 min at max. g. 200 μl of the supernatant was transferred to an HPLC vial with an insert and measured by HPLC (RI detection). For the HPAEC measurements (conductivity detection), the clear supernatant was diluted 1:250.

After 16 h, only allitol and D-gluconate could be detected in the reactor.

The entire reactor contents were then heated to 70° C. for 60 minutes (deactivation of D-psicose 3-epimerase) and, after cooling to 30° C., 17.5 ml of xylitol dehydrogenase lysate, 7 kU of NADH oxidase lysate, and 3.5 ml of a 10 mM NAD+ solution were added.

Allitol was completely oxidized to D-psicose within 27 h.

In this way, 17.5 g of D-fructose could be oxidized 100% to D-psicose (17.8 g in solution). D-sorbitol and D-fructose could not be detected in the resulting solution.

The reactor contents were heated to 70° C., the pH was adjusted to 4, and the mixture was stirred for 30 min at 70° C. The enzymes were filtered using a glass frit (P3).

### Example 3

Production of D-Psicose from D-Fructose—Cofactor Regeneration with FDH and Sodium Formate

The reaction was carried out in a Multifors tabletop bioreactor (Infors AG). A glass reactor (volume 1 l) with an agitator and a pH electrode was used as vessel. The pH was controlled by adding 5M NaOH or 6M H₂SO₄.

At the start, 70 ml of a D-fructose solution (500 g/l), 26.3 ml of deionized water, and 43.8 ml of an 8 M sodium formate solution were placed in the reactor and heated to 37° C. with stirring.

To start the reaction, 25 ml of D-psicose-3-epimerase lysate was added. Then 35 ml of SDR I lysate, 7 kU of formate dehydrogenase lysate, and 17.5 ml of a 10 mM NAD⁺ solution were added.

During operation, samples were continuously taken from the reactor solution and analyzed as follows: 100 μl of the reactor solution was mixed with 200 μl of methanol and incubated in an Eppendorf Thermomixer at 60° C. and 1200 rpm for 15 min. The sample was briefly centrifuged, mixed with 700 μl of deionized water, vortexed, and then centrifuged for 5 min at max. g. 200 μl of the supernatant was transferred to an HPLC vial with an insert and measured by HPLC (RI detection).

After 40 h, 95% of the D-fructose (100 g/l) was converted to allitol.

The entire reactor contents were then heated to 70° C. for 60 min (deactivation of D-psicose 3-epimerase) and, after cooling to 24° C., 25 ml of xylitol dehydrogenase lysate, 10 kU of NADH oxidase lysate, and 10 ml of a 10 mM NAD⁺ solution were added.

Allitol was completely oxidized to D-psicose within 4 h. The reactor contents were heated to 70° C., the pH was adjusted to 4, and the mixture was stirred for 30 minutes at 70° C. The enzymes were filtered using a glass frit (P3).

In this way, 97% of the D-fructose was converted to D-psicose. Traces of D-fructose were still detected in the reaction solution, but no D-sorbitol.

### LITERATURE

Zhang, W., Yu, S., Zhang, T., Jiang, B., & Mu, W. (2016). Recent advances in D-allulose: Physiological functionalities, applications, and biological production. Trends in Food Science and Technology, 54, 127-137. https://doi.org/10.1016/j.tifs.2016.06.004
Hough, L., & Stacey, B. E. (1996). Variation in the allitol content of Itea plants during photosynthesis. Phytochemistry, 5(1), 171-175. https://doi.org/10.1016/S0031-9422(00)85095-5
Oshima, H., Kimura, I., & Izumori, K. (2006). Psicose Contents in Various Food Products and its Origin. Food Science and Technology Research, 12(2), 137-143. https://doi.org/10.3136/fstr.12.137
Jiang, S., Xiao, W., Zhu, X., Yang, P., Zheng, Z., Lu, S., Jiang, S., Zhang, G., & Liu, J. (2020). Review on D-Allulose: In vivo Metabolism, Catalytic Mechanism, Engineering Strain Construction, Bio-Production Technology. Frontiers in Bioengineering and Biotechnology, 8, 26. https://doi.org/10.3389/fbioe.2020.00026
Ahmed, A., Khan, T. A., Ramdath, D. D., Kendall, C. W. C., & Sievenpiper, J. L. (2022). Rare sugars and their health effects in humans: a systematic review and narrative synthesis of the evidence from human trials, Nutrition Reviews, 80(2), 255-270. https://doi.org/10.1093/nutrit/nuab012
Chen, Z., Gao, X.-D., & Li, Z. (2022). Recent Advances Regarding the Physiological Functions and Biosynthesis of D-Allulose. Frontiers in Microbiology, 13, 881037. https://doi.org/10.3389/fmicb.2022.881037
Doner, L. W. (1979). Isomerization of D-fructose by base: Liquid-chromatographic evaluation and the isolation of D-psicose. Carbohydrate Research, 70(2), 209-216. https://doi.org/10.1016/S0008-6215(00)87101-3
Bilik, V., & Tihlarik, K. (1974). Reactions of saccharides catalyzed by molybdate ions. IX. Epimerization of ketohexoses. Chemical Papers, 28(1), 106-109. https://www.chemicalpapers.com/?id=7&paper=5533
Izumori, K., Khan, A. R., Okaya, H., & Tsumura, T. (1993). A New Enzyme, D-Ketohexose 3-Epimerase, from Pseudomonas sp. ST-24. Bioscience, Biotechnology, and Biochemistry, 57(6), 1037-1039. https://doi.org/10.1271/bbb.57.1037
Kim, N.-H., Kim, H.-J., Kang, D.-I., Jeong, K.-W., Lee, J.-K., Kim, Y., & Oh, D.-K. (2008). Conversion Shift of D-Fructose to D-Psicose for Enzyme-Catalyzed Epimerization by Addition of Borate. Applied and Environmental Microbiology, 74(10), 3008-3013. https://doi.org/10.1128/AEM.00249-08
Lim, B.-C., Kim, H.-J., & Oh, D.-K. (2009). A stable immobilized D-psicose 3-epimerase for the production of D-psicose in the presence of borate. Process Biochemistry, 44(8), 822-828. https://doi.org/10.1016/j.procbio.2009.03.017
Zhu, P., Zeng, Y., Chen, P., Men, Y., Yang, J., Yue, X., Zhang, J., Zhu, Y., & Sun, Y. (2020). A one-pot two-enzyme system on the production of high value-added D-allulose from Jerusalem artichoke tubers. Process Biochemistry, 88, 90-96. https://doi.org/10.1016/j.procbio.2019.10.006
Li, C. Li, L., Feng, Z., Guan, L., Lu, F., & Qin, H.-M. (2021). Two-step biosynthesis of D-allulose via a multienzyme cascade for the bioconversion of fruit juices. Food Chemistry, 357, 129746. https://doi.org/10.1016/j.foodchem.2021.129746
Juneja, A., Zhang, G., Jin, Y.-S., & Singh, V. (2019). Bioprocessing and technoeconomic feasibility analysis of simultaneous production of D-psicose and ethanol using engineered yeast strain KAM-2GD. Bioresource Technology, 275, 27-34. https://doi.org/10.1016/j.biortech.2018.12.025
Patel, S. N., Singh, V., Sharma, M., Sangwan, R. S., Singhal, N. K., & Singh, S. P. (2018). Development of a thermo-stable and recyclable magnetic nanobiocatalyst for bioprocessing of fruit processing residues and D-allulose synthesis. Bioresource Technology, 247, 633-639. https://doi.org/10.1016/j.biortech.2017.09.112
Yang, P., Zhu, X., Zheng, Z., Mu, D., Jiang, S., Luo, S., Wu, Y., & Du, M. (2018). Cell regeneration and cyclic catalysis of engineered Kluyveromyces marxianus of a D-psicose-3-epimerase gene from Agrobacterium tumefaciens for D-allulose production. World Journal of Microbiology and Biotechnology, 34, 65. https://doi.org/10.1007/si1274-018-2451-6
Dedania, S. R., Patel, V. R., Soni, S. S., & Patel, D. H. (2020). Immobilization of Agrobacterium tumefaciens D-psicose 3-epimerase onto titanium dioxide for bioconversion of rare sugar. Enzyme and Microbial Technology, 140, 109605. https://doi.org/10.1016/j.enzmictec.2020.109605
Van Duc Long, N., Le, T.-H., Kim, J.-I., Lee, J. W. and Koo, Y.-M. (2009). Separation of D-psicose and D-fructose using simulated moving bed chromatography. Journal of Separation Science, 32(11), 1987-1995. https://doi.org/10.1002/jssc.200800753
Li, Y., Shi, T., Han, P., & You, C. (2021). Thermodynamics-Driven Production of Value-Added D-Allulose from Inexpensive Starch by an In Vitro Enzymatic Synthetic Biosystem. ACS Catalysis, 11(9), 5088-5099. https://doi.org/10.1021/acscatal.0c05718
Wang, W., Yang, J., Sun, Y., Li, Z., & You, C. (2020). Artificial ATP-Free in Vitro Synthetic Enzymatic Biosystems Facilitate Aldolase-Mediated C—C Bond Formation for Biomanufacturing. ACS Catalysis, 10(2), 1264-1271. https://doi.org/10.1021/acscatal.9b04696
Li, Z., Li, F., Cai, L., Chen, Z., Qin, L., & Gao, X.-D. (2020). One-Pot Multienzyme Synthesis of Rare Ketoses from Glycerol. Journal of Agricultural and Food Chemistry, 68(5), 1347-1353. https://doi.org/10.1021/acs.jafc.9b06748
Xiao, Q., Niu, J., Liu, H., Liu, Y., & Zhou, X. (2019). High Conversion of D-Fructose into D-Allulose by Enzymes Coupling with an ATP Regeneration System. Molecular Biotechnology, 61, 432-441. https://doi.org/10.1007/s12033-019-00174-6
Zhang, J., Luo, W., Wang, Z., Chen, X., Lv, P., & Xu, J. (2021). A novel strategy for D-psicose and lipase co-production using a co-culture system of engineered Bacillus subtilis and Escherichia coli and bioprocess analysis using metabolomics. Bioresources and Bioprocessing, 8, 77. https://doi.org/10.1186/s40643-021-00429-8
Takeshita, K., Ishida, Y., Takada, G., & Izumori, K. (2000). Direct Production of Allitol from D-Fructose by a Coupling Reaction Using D-Tagatose 3-Epimerase, Ribitol Dehydrogenase and Formate Dehydrogenase. Journal of Bioscience and Bioengineering, 90(5), 545-548. https://doi.org/10.1016/S1389-1723(01)80038-4
Gullapalli, P., Takata, G., Poonperm, W., Rao, D., Morimoto, K., Akimitsu, K., Tajima, S., & Izumori, K. (2007). Bioproduction of D-psicose from allitol with Enterobacter aerogenes IK7: a new frontier in rare ketose production. Bioscience, Biotechnology, and Biochemistry, 71(12), 3048-3054. https://doi.org/10.1271/bbb.70450
Poonperm, W., Takata, G., Ando, Y., Sahachaisaree, V., Lumyong, P., Lumyong, S., & Izumori, K. (2007). Efficient conversion of allitol to D-psicose by Bacillus pallidus Y25. Journal of Bioscience and Bioengineering, 103(3), 282-285. https://doi.org/10.1263/jbb.103.282
Izumori, K. (2006). Izumoring: a strategy for bioproduction of all hexoses. Journal of Biotechnology, 124(4), 717-722. https://doi.org/10.1016/j.jbiotec.2006.04.016
Hassanin, H. A. M., Mu, W., Koko, M. Y. F., Zhang, T., Masamba, K., Jiang, B. (2017). Allitol: production, properties and applications. International Journal of Food Science and Technology, 52(1), 91-97. https://doi.org/10.1111/ijfs.13290
Hold, C., & Panke, S. (2009). Towards the engineering of in vitro systems. Journal of the Royal Society Interface, 6(4), S507-S521. https://doi.org/10.1098/rsif.2009.0110.focus
Siedentop, R., ClaaBen, C., Rother, D., Ltz, S., & Rosenthal, K. (2021). Getting the Most Out of Enzyme Cascades: Strategies to Optimize In Vitro Multi-Enzymatic Reactions. Catalysts, 11(10), 1183. https://doi.org/10.3390/catal11101183
Wang, L., Hu, M., & Tao, Y. (2022). Efficient production of D-glucosamine by diacetylchitobiose deacetylase catalyzed deacetylation of N-acetyl-D-glucosamine. Biotechnology Letters, 44, 473-483. https://doi.org/10.1007/s10529-022-03225-2
Wang, L., Chen, K., Zheng, P., Huo, X., Liao, F., Zhu, L., Hu, M., & Tao, Y. (2023). Enhanced production of D-psicose from D-fructose by a redox-driven multi-enzyme cascade system. Enzyme and Microbial Technology, 163, 110172. https://doi.org/10.1016/j.enzmictec.2022.110172
Xu, J., Zhou, H., Yu, H., Deng, T., Wang, Z., Zhang, H., Wu, J., & Yang, L. (2021). Computational design of highly stable and soluble alcohol dehydrogenase for NADPH regeneration. Bioresources and Bioprocessing, 8, 12. https://doi.org/10.1186/s40643-021-00362-w
Al-Rabiah, A. A., Boz, I., Akhmedov, V. M., Mostafa, M. M. M., & Bagabas, A. A. (2022). Highly Selective Gas-Phase Catalytic Hydrogenation of Acetone to Isopropyl Alcohol. Catalysts, 12(10), 1251. https://doi.org/10.3390/catal12101251
Lin, B., & Tao, Y. (2017). Whole-cell biocatalysts by design. Microbial Cell Factories, 16, 106. https://doi.org/10.1186/s12934-017-0724-7
Kawakami, K., Hasegawa, Y., Deguchi, K., Ohki, S., Shimizu, T., Yoshihashi, Y., Yonemochi, E., & Terada, K. (2013). Competition of Thermodynamic and Dynamic Factors During Formation of Multicomponent Particles via Spray Drying. Journal of Pharmaceutical Sciences, 102(2), 518-529. https://doi.org/10.1002/jps.23378
Kawakami, K., Hasegawa, Y., Zhang, S., Yoshihashi, Y., Yonemochi, E., & Terada, K. (2014). Low-Density Microparticles with Petaloid Surface Structure for Pulmonary Drug Delivery. Journal of Pharmaceutical Sciences, 103(4), 1309-1313. https://doi.org/10.1002/jps.23899
Altschul, S. F., Gish, W., Miller, W., Myers, E. W., & Lipman, D. J. (1990). Basic local alignment search tool. Journal of Molecular Biology, 215(3), 403-410. https://doi.org/10.1016/S0022-2836(05)80360-2
Henikoff, S., & Henikoff, J. G. (1992). Amino acid substitution matrices from protein blocks. Proceedings of the National Academy of Sciences of the United States of America, 89(22), 10915-10919. https://doi.org/10.1073/pnas.89.22.10915
Sambrook, J., Fritsch, E. R., & Maniatis, T. (1989). Molecular Cloning: A Laboratory Manual (2nd ed.). Cold Spring Harbor, NY: Cold Spring Harbor Laboratory Press.
Mu, W., Chu, F., Xing, Q., Yu, S., Zhou, L., & Jiang, B. (2011). Cloning, Expression, and Characterization of a D-Psicose 3-Epimerase from Clostridium cellulolyticum H10. Journal of Agricultural and Food Chemistry, 59(14), 7785-7792. https://doi.org/10.1021/jf201356q
Chan, H.-C., Zhu, Y., Hu, Y., Ko, T.-P., Huang, C.-H., Ren, F., Chen, C.-C., Ma, Y., Guo, R.-T., & Sun, Y. (2012). Crystal structures of D-psicose 3-epimerase from Clostridium cellulolyticum H10 and its complex with ketohexose sugars. Protein & Cell, 3(2), 123-131. https://doi.org/10.1007/s13238-012-2026-5
Protein [Internet]. Bethesda (MD): National Library of Medicine (US), National Center for Biotechnology Information. Accession No. WP_063903495.1, SDR family oxidoreductase [Gluconobacter frateurii]. Available at: https://www.ncbi.nlm.nih.gov/protein/WP_063903495.1/(Accessed on 7 Mar. 2024)
Habenicht, A., Motejadded, H., Kiess, M., Wegerer, A., & Mattes, R. (1999). Xylose Utilisation: Cloning and Characterisation of the Xylitol Dehydrogenase from Galactocandida mastotermitis. Biological Chemistry, 380(12), 1405-1411. https://doi.org/10.1515/BC.1999.180
Protein [Internet]. Bethesda (MD): National Library of Medicine (US), National Center for Biotechnology Information. Accession No. WP_013084280.1, NAD(P)-dependent alcohol dehydrogenase [Priestia megaterium]. Available at: https://www.ncbi.nlm.nih.gov/protein/WP_013084280.1/(Accessed on 7 Mar. 2024)
Protein [Internet]. Bethesda (MD): National Library of Medicine (US), National Center for Biotechnology Information. Accession No. WP_070401870.1, SDR family oxidoreductase [Kozakia baliensis]. Available at: https://www.ncbi.nlm.nih.gov/protein/WP_070401870.1/ (Accessed on 7 Mar. 2024)
Protein [Internet]. Bethesda (MD): National Library of Medicine (US), National Center for Biotechnology Information. Accession No. WP_068907433.1, SDR family oxidoreductase [Providencia heimbachae]. Available at: https://www.ncbi.nlm.nih.gov/protein/WP_068907433.1/ (Accessed on 7 Mar. 2024)
Sakoda, H., & Imanaka, T. (1992). Cloning and sequencing of the gene coding for alcohol dehydrogenase of Bacillus stearothermophilus and rational shift of the optimum pH. Journal of Bacteriology, 174(4), 1397-1402. https://doi.org/10.1128/jb.174.4.1397-1402.1992
Matsumoto, J., Higuchi, M., Shimada, M., Yamamoto, Y., & Kamio, Y. (1996). Molecular Cloning and Sequence Analysis of the Gene Encoding the H₂O-forming NADH Oxidase from Streptococcus mutans. Bioscience, Biotechnology, and Biochemistry, 60(1), 39-43. https://doi.org/10.1271/bbb.60.39
Protein [Internet]. Bethesda (MD): National Library of Medicine (US), National Center for Biotechnology Information. Accession No. MDQ0804260.1, Glucose 1-dehydrogenase [Priestia megaterium]. Available at: https://www.ncbi.nlm.nih.gov/protein/MDQ0804260.1 (Accessed on 7 Mar. 2024)
Protein [Internet]. Bethesda (MD): National Library of Medicine (US), National Center for Biotechnology Information. Accession No. WP_013168047.1, NAD-dependent formate dehydrogenase [Ancylobacter novellus]. Available at: https://www.ncbi.nlm.nih.gov/protein/WP 013168047.1 (Accessed on 7 Mar. 2024)

1. A method for preparing an aqueous solution containing D-psicose by forming a first D-psicose from a D-fructose, which is present in an aqueous solution, by treatment with an epimerase in vitro, after which the first D-psicose is reduced to allitol by treatment with a respective NAD(P)H-dependent oxidoreductase in vitro and, after deactivation and/or ultrafiltration of the epimerase, added a respective NAD(P)⁺-dependent oxidoreductase for forming D-psicose, after which the deactivated epimerase and the oxidoreductases are removed.

2. The method according to claim 1, wherein the NAD(P)⁺-dependent oxidoreductase for the formation of D-psicose from allitol comprises an amino acid sequence selected from the group consisting of:
i) an amino acid sequence having at least 80% identity to SEQ ID NO: 4, SEQ ID NO: 6, SEQ ID NO: 8, SEQ ID NO: 10, or SEQ ID NO: 12,
ii) an amino acid sequence encoded by a nucleic acid having at least 80% identity to SEQ ID NO: 3, SEQ ID NO: 5, SEQ ID NO: 7, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 5, SEQ ID NO: 7, SEQ ID NO: 9, or SEQ ID NO: 11.

3. The method according to claim 1, wherein the oxidized co-factor NAD(P)⁺ formed by the reduction is reduced by means of an alcohol dehydrogenase and a secondary alcohol with the formation of a ketone.

4. The method according to claim 3, wherein the secondary alcohol is D-glucose or 2-propanol.

5. The method according to claim 1, wherein the method is carried out as a one-pot reaction without isolation of any intermediate products.

6. The method according to claim 1, wherein the enzymes are present as a lysate of the corresponding cells producing them.

7. The method according to claim 1, wherein the NAD(P)H-dependent oxidoreductase for the reduction of the first D-psicose to allitol comprises an amino acid sequence selected from the group consisting of:
i) an amino acid sequence having at least 80% identity to SEQ ID NO: 4, SEQ ID NO: 10, or SEQ ID NO: 12,
ii) an amino acid sequence encoded by a nucleic acid having at least 80% identity to SEQ ID NO: 3, SEQ ID NO: 9, or SEQ ID NO: 11, and
iii) an amino acid sequence encoded by a nucleic acid that, under stringent conditions, binds to a nucleic acid molecule comprising the nucleic acid sequence of SEQ ID NO: 3, SEQ ID NO: 9, or SEQ ID NO: 11.

8. The method according to claim 3, wherein the alcohol dehydrogenase comprises or consists of an amino acid sequence selected from the group consisting of:
i) an amino acid sequence having at least 80% identity to SEQ ID NO: 18,
ii) an amino acid sequence encoded by a nucleic acid having at least 80% identity to SEQ ID NO: 17, and
iii) an amino acid sequence encoded by a nucleic acid that, under stringent conditions, binds to a nucleic acid molecule comprising the nucleic acid sequence of SEQ ID NO: 17.

9. The method according to claim 1, wherein the oxidized co-factor NAD(P)⁺ formed by the reduction is reduced by means of a glucose dehydrogenase and D-glucose with the formation of D-gluconate.

10. The method according to claim 9, wherein the glucose dehydrogenase comprises or consists of an amino acid sequence selected from the group consisting of:
i) an amino acid sequence having at least 80% identity to SEQ ID NO: 20,
ii) an amino acid sequence encoded by a nucleic acid having at least 80% identity to SEQ ID NO: 19, and
iii) an amino acid sequence encoded by a nucleic acid that, under stringent conditions, binds to a nucleic acid molecule comprising the nucleic acid sequence of SEQ ID NO: 19.

11. The method according to claim 1, wherein the oxidized cofactor NAD(P)⁺ produced by the reaction is reduced by means of a formate dehydrogenase and formate with the formation of CO₂.

12. The method according to claim 11, wherein the formate dehydrogenase comprises or consists of an amino acid sequence an amino acid sequence selected from the group consisting of:
i) an amino acid sequence having at least 80% identity to SEQ ID NO: 2,
ii) an amino acid sequence encoded by a nucleic acid having at least 80% identity to SEQ ID NO: 1, and
iii) an amino acid sequence encoded by a nucleic acid that, under stringent conditions, binds to a nucleic acid molecule comprising the nucleic acid sequence of SEQ ID NO: 1.

13. A method of manufacturing a syrup containing D-psicose comprising concentrating the aqueous solution preparable by the method according to claim 1.

14. (canceled)

15. (canceled)

16. (canceled)

17. (canceled)

18. The method according to claim 1, further comprising using an H₂O-forming NAD(P)H oxidase for co-factor regeneration comprising or consisting of an amino acid sequence selected from the group consisting of:
i) an amino acid sequence having at least 80% identity to SEQ ID NO: 14 or SEQ ID NO: 16,
ii) an amino acid sequence encoded by a nucleic acid having at least 80% identity to SEQ ID NO: 13 or SEQ ID NO: 15, and
iii) an amino acid sequence encoded by a nucleic acid that, under stringent conditions, binds to a nucleic acid molecule comprising the nucleic acid sequence of SEQ ID NO: 13 or SEQ ID NO: 15.


SEQUENCE LISTING (20 sequences)

SEQ ID NO: 1 - DNA, 1206 bp, Starkeya novella
  source 1..1206: mol_type = genomic DNA
atggcgaaaatactttgcgttctctatgacgatccggtcgacggctacccgaagacctatgcgcgcgacgacctgccgaagatcgaccactatccgggcgggcagacgctgcccacgcccaaggcgatcgacttcacgccgggcgcgctgctcggctcggtctccggcgagctcggcctgcgcaaatacctggaagccaacggccataccttcgtcgtcacctccgataaggacggcccggattcggtgttcgagagggaactcgtcgacgccgacgtggtgatctcgcagcccttctggccggcctatctgacgcccgagcgcatcgccaaggcgaagaacctgaagctcgcgctcaccgccggcatcggctccgatcatgtcgatcttcagtcagctatcgaccgtggcatcactgtggccgaagtcacatattgcaactcgatcagcgtcgccgagcacgtggtgatgatgatcctcggcctggtacgaaactacattccctcgcatgactgggcgcgcaagggcggctggaacatagccgactgcgtagagcactcctacgacctcgagggcatgaccgtcggctcggtggccgccggccgcatcggcctcgccgtgctgcgccgcctcgcgccgttcgacgtgaagctgcactataccgaccgccaccgtctgccagaagcggtcgagaaggagctgggcctcgtctggcacgatacccgcgaggacatgtacccgcattgcgacgtggtcacgctcaacgtgccgctgcaccccgaaaccgagcacatgatcaatgacgagacgctgaagctgttcaagcgcggcgcctatatcgtcaacaccgcccgcggcaagctcgccgaccgcgacgccatcgtccgcgcgatcgagagcgggcagctcgcgggctatgccggcgacgtgtggttcccgcagccggctccgaaggaccacccctggcgcaccatgaagtgggaaggcatgacgccgcacatctccggcacctcgctctctgcccaggcgcgctacgcggcgggcacgcgcgagatcctcgaatgcttcttcgagggccggccgatccgcgacgagtacctgatcgtgcagggcggcgcgctcgccggcaccggcgcgcattcctactcgaagggcaatgcgaccggcggttcggaagaggccgcgaagttcaagaaggctggctga

SEQ ID NO: 2 - protein, 401 aa, Starkeya novella
MAKILCVLYDDPVDGYPKTYARDDLPKIDHYPGGQTLPTPKAIDFTPGALLGSVSGELGLRKYLEANGHTFVVTSDKDGPDSVFERELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 3 - DNA, 785 bp, Gluconobacter frateurii
  source 1..785: mol_type = genomic DNA
atgacacagtctcttcagggcaagatcgtcgccattactggcgcggcttcgggcattggcctcgaatgcgcccgctatctcatcgaagctggcgcggtggtctatcttctggaccgtgacgccaaaactctcgaagacaaaacggcagaactcggcagccaggcccatgcgatcatcgtcgatctcttcgactacaaaaccgtagatgctgcggtcgcgcagatcgttgaggagcagggccggatcgatgttttccacgccaacgccggcgcgtatgtgggcggcaatgtctgggaaggggatcccgatagctgggatgcgatgctgcacctgaacatcaatgcagctttccgctcggtccgtgccgtgctgccgcagatgatgaagcaggaaagtggcgatatcgtcatgaccagctcgatcgcgggcatgatccccatcatggccgagccgatctacacggcctcgaaacatgccgtgcaggcattcgtacatacggtgcgccgacaggtcgcgaagtatggaattcgtgtgggtgcaatccagcctggtcctgtagtcacgcctttgctgaaagactgggatcaggcccgtcttgaagccaacatcaaagcgggtgccctgatggaagccaaggaagtcgccgaagccctgatcttcatcctgacacgttcaaagggtgtcatggtgcgggatctggtcgttctgccacataacttcgacgcctaagcttaagcggccgcactcgagcaccaccaccaccaccactgagatccggctgctaa

SEQ ID NO: 4 - protein, 242 aa, Gluconobacter frateurii
MTQSLQGKIVAITGAASGIGLECARYLIEAGAVVYLLDRDAKTLEDKTAELGSQAHAIIVDLFDYKTVDAAVAQIVEEQGRIDVFHANAGAYVGGNVWEGDPDSWDAMLHLNINAAFRSVRAVLPQMMKQESGDIVMTSSIAGMIPIMAEPIYTASKHAVQAFVHTVRRQVAKYGIRVGAIQPGPVVTPLLKDWDQARLEANIKAGALMEAKEVAEALIFILTRSKGVMVRDLVVLPHNFDA

SEQ ID NO: 5 - DNA, 1062 bp, Galactocandida mastotermitis
  source 1..1062: mol_type = genomic DNA
atgagcacaccggaaaatctgagctttgtgctgcagaaaccgtttgatgtgaaatttgaagatcgtccgattccgaaactgagcgatccgtatagcgttaaaattcaggtgaaaaaaaccggcatttgcggtagtgatgttcactatttcacccatggtgcaattggtgattttgttgttaaagcaccgatggttctgggtcatgaaagcagcggtgttgttctggaagttggtagcgaagttaaaagcctgaaagttggtgatcgtgttgcaatggaaccgggtgttccgagccgtcatagtgatgagtataaaagcggtcgttataatctgtgtccgcacatggcatttgcagcaacccctccgtatgatggcaccctgtgtaaatactatattctgccggaagatttctgcgttaaactgccggaacatgttagcctggaagaaggtgcactggttgaaccgctgagcgttgcagttcatagcagcaaactgggtaacattaaaccgggtagccatgttgcaatttatggtgcaggtccggttggtctgctggttgcagcagttgcaagcgcatttggtgcagaaagcgttaccattattgatctggttgaaagccgtctgaatctggcaaaagaactgggtgcaaccgcaaccgttcaggttgattttaaagataccccgaaagaaagcgcagcaaaagttgttgcagcaaataatggcattgcaccggatgttgttattgatgcaagcggtgcagaagcaagcattaattcagccattaatgcaattcgtccgggtggcacctatgttcaggtgggtatgggtaaaccggatgtgagctttccgattgcaaccctgattggtaaagaactgaccgttaaaggtagctttcgttatggttatggtgattatccgctggcagttagcctgctggcaagcggtaaagttaatgtgaaaaaactgatcacccatgaagtgaaattcgaggatgcagcagaagcatttcagctggttcgtgatggtaaagccattaaatgtattatcaacggtccggaataa

SEQ ID NO: 6 - protein, 353 aa, Galactocandida mastotermitis
MSTPENLSFVLQKPFDVKFEDRPIPKLSDPYSVKIQVKKTGICGSDVHYFTHGAIGDFVVKAPMVLGHESSGVVLEVGSEVKSLKVGDRVAMEPGVPSRHSDEYKSGRYNLCPHMAFAATPPYDGTLCKYYILPEDFCVKLPEHVSLEEGALVEPLSVAVHSSKLGNIKPGSHVAIYGAGPVGLLVAAVASAFGAESVTIIDLVESRLNLAKELGATATVQVDFKDTPKESAAKVVAANNGIAPDVVIDASGAEASINSAINAIRPGGTYVQVGMGKPDVSFPIATLIGKELTVKGSFRYGYGDYPLAVSLLASGKVNVKKLITHEVKFEDAAEAFQLVRDGKAIKCIINGPE

SEQ ID NO: 7 - DNA, 1062 bp, Priestia megaterium
  source 1..1062: mol_type = genomic DNA
atgaataacaacctgccgaaaaccatgaaagcagcagttatgcatggcacccgtgaaattagcattgaaaccctgccgattccgcagattgatgaaaatgaagttctgatcaaagttatggccgttggtatttgtggtagcgatctgcactattacacccagggtcgtattggtaaatacaaagtggaaaaaccgtttatcctgggtcatgaatgtagcggtgaagttgttgcaattggtagcgcagttgaacgttttcgtgttggtgatcgtgttgccgttgaaccgggtgttacctgtggtcattgtgaagcatgtaaagagggtcgttataatctgtgtccggatgttcagtttctggcaacccctccggttgatggtgcatttgttcagtatatcaaaatgcgccaggatttcgtttttctgattccgaatagcctgagctatgaagatgcagcactgattgaaccgtttagcgtgggtattcatgcagcaacccgtaccaaactgcagcctggtagcaccattgcaattatgggtatgggtccggttggtctgatggcagttgcagcagcaaaagcatttggtgcaagcaccattattgcaaccgatctggaaccgctgcgtctggaagcagccaaacgtatgggtgcaacccatgttattaacattcgtgaacaggatccgctgaacgagattaaaaacattaccgaaaatgtgggtgttgatgttgcatgggaaaccgcaggtaatccgaaagcactgcagagcagcctgagcagcattcgtcgtggtggtaaactggcaattgttggtctgccgagccagagcgatattccgctggatgttccgtttattgccgataatgaaatcgatatctatggcatctttcgctatgcaaacacctatccgaaaggcatcaaatttctgaccagcggtgcaattgataccaaaaatctggttaccgatcgttatccgctggcaggtacacgtgaagcaatggaacgtgcactgaatttcaaaaacgaatgcctgaaaatcatcgtgtatccgaacgaataa

SEQ ID NO: 8 - protein, 353 aa, Priestia megaterium
MNNNLPKTMKAAVMHGTREISIETLPIPQIDENEVLIKVMAVGICGSDLHYYTQGRIGKYKVEKPFILGHECSGEVVAIGSAVERFRVGDRVAVEPGVTCGHCEACKEGRYNLCPDVQFLATPPVDGAFVQYIKMRQDFVFLIPNSLSYEDAALIEPFSVGIHAATRTKLQPGSTIAIMGMGPVGLMAVAAAKAFGASTIIATDLEPLRLEAAKRMGATHVINIREQDPLNEIKNITENVGVDVAWETAGNPKALQSSLSSIRRGGKLAIVGLPSQSDIPLDVPFIADNEIDIYGIFRYANTYPKGIKFLTSGAIDTKNLVTDRYPLAGTREAMERALNFKNECLKIIVYPNE

SEQ ID NO: 9 - DNA, 729 bp, Kozakia baliensis
  source 1..729: mol_type = genomic DNA
atgacctctcctctccagggtaagatagccgccatcacgggcggggcttcgggcatcggcctcgaatgtgtccgccagatcgccgcaagtggtgccacggtttatattctcgaccgcgaccatcaggcgctcgacaaggcgcgcgaagaattgggcgagcgcgttcataccatcgaggtcgatctcttccgttacgaaacggtcgatcgcgccatcgaaaccatcgtgtccgaacaaggacgcatcgacattctccatgtcaatgcgggcgcgtatatcggcggcaatgtctgggaaggcgatcccgataaatgggacaagatgctgaatctcaacatcaacgccgccttccgttccgcccgcgccgtcatgcccgccatgatgaagcagaaaagcggcgatatcatcatgacaagctcgatcgcaggcatcgtcccgatcccggcggagccgatctacacggcttccaaacatgcggtgcaagccttcgctcacaccatacgccgccagttggccccgttcggcatccgcgtcggcgccatccagcccggcccggtcgtcacgcccttgctcaatgattgggaccccgagcgccttaaagccaatatcgaggctggcgccatgatgcagccttctgacgtcgccgaagccgtggttttcatgctgtcccgccgcaagggaacggtaatccgcgacttggttctgttaccccattctttcgacgtctaa

SEQ ID NO: 10 - protein, 242 aa, Kozakia baliensis
MTSPLQGKIAAITGGASGIGLECVRQIAASGATVYILDRDHQALDKAREELGERVHTIEVDLFRYETVDRAIETIVSEQGRIDILHVNAGAYIGGNVWEGDPDKWDKMLNLNINAAFRSARAVMPAMMKQKSGDIIMTSSIAGIVPIPAEPIYTASKHAVQAFAHTIRRQLAPFGIRVGAIQPGPVVTPLLNDWDPERLKANIEAGAMMQPSDVAEAVVFMLSRRKGTVIRDLVLLPHSFDV

SEQ ID NO: 11 - DNA, 729 bp, Providencia heimbachae
  source 1..729: mol_type = genomic DNA
atggctatatctctcgaaaacaacgtagctgcaattacaggtgccgcttcaggtatcggtctcgaatgtgcacgcacactgatcaaagcaggcgctaaagttgtcctcattgaccgagcagaagatagactaaatcaattggtcgcagaattaggtgaaaatgcaattccattagttatcgatttaatgaaaccagaacaagtcgatggcatgttagcgcgtattatcgaaaaggcaggcagattagatatctttcatgctaatgctggagcttacattggtgggcccgtagccgaaggcgatcccgatgtttgggataaagtcctaaatttaaatgttaatgccgcattccgctgtgttcgcgcagttctaccacactttatcgcacaaaagtcaggcgatattctattcaccagctctatcgctggtatggttcccgtaatttgggagcctatttacacggcatcaaaatttgcggttcaagcattcgttcattctactcgccgtcaggtttctgaacacggtgtccgtgttggtgctgtattacctggtcctgttgttactgcgttattagatgattggccaaaagaaaaacttgaagaagctttagctaacggtagtttaatgcaacccattgaagttgctgaggctgttctattcatgctgacgcgtccaagaaatattacaattcgcgatttagttattttacccaatagtgttgacctctaa

SEQ ID NO: 12 - protein, 242 aa, Providencia heimbachae
MAISLENNVAAITGAASGIGLECARTLIKAGAKVVLIDRAEDRLNQLVAELGENAIPLVIDLMKPEQVDGMLARIIEKAGRLDIFHANAGAYIGGPVAEGDPDVWDKVLNLNVNAAFRCVRAVLPHFIAQKSGDILFTSSIAGMVPVIWEPIYTASKFAVQAFVHSTRRQVSEHGVRVGAVLPGPVVTALLDDWPKEKLEEALANGSLMQPIEVAEAVLFMLTRPRNITIRDLVILPNSVDL

SEQ ID NO: 13 - DNA, 1374 bp, unidentified
  source 1..1374: mol_type = genomic DNA
atgagcaaaattgttatcgtgggtgcaaatcatgcaggcaccgcagcaattaataccattctggataattatggcagcgaaaatgaagtggttgtgtttgatcagaatagcaacattagctttctgggttgtggtatggcactgtggattggtaaacaaattagcggtccgcagggtctgttttatgcagataaagaaagcctggaagcaaaaggtgccaaaatctatatggaaagtccggttaccgccattgattatgatgcaaaacgtgttaccgcactggttaatggtcaagaacatgttgaaagctacgagaaactgattctggcaaccggtagcaccccgattctgcctccgattaaaggtgcagccattaaagaaggtagtcgcgattttgaagcaaccctgaaaaatctgcagttcgtgaaactgtatcagaatgccgaagatgtgattaacaaactgcaggataaaagccagaatctgaatcgtattgcagttgttggtgcaggttatattggtgttgaactggcagaagcatttaaacgtctgggtaaagaagtgattctgattgacgttgttgatacctgtctggcaggttattatgatcaggatctgagcgaaatgatgcgtcagaatctggaagatcatggtatcgaactggcatttggtgaaaccgttaaagcaattgaaggtgatggtaaagtggaacgtattgttaccgataaagcaagccatgatgtggatatggttattctggcagttggttttcgtccgaatacagcactgggtaatgcaaaactgaaaacctttcgtaatggtgcctttctggtggataaaaaacaagaaaccagcatcccggatgtttatgcaattggtgattgtgcaaccgtgtatgataatgccattaacgacaccaactatattgcactggcaagcaatgcactgcgtagcggtattgttgcaggtcataatgcagccggtcataaactggaaagtctgggtgttcagggtagcaatggtatttcaatttttggcctgaatatggttagcaccggtctgacccaagaaaaagccaaacgttttggttataatccggaagttaccgcctttaccgattttcagaaagccagctttatcgagcatgataactatccggttacgctgaaaattgtgtatgacaaagatagccgtctggttctgggtgcacagatggccagcaaagaagatatgagcatgggtattcacatgtttagcctggccattcaagagaaagttaccattgaacgtctggccctgctggattatttctttctgccgcattttaatcagccgtacaactatatgaccaaagcagcactgaaagccaaataa

SEQ ID NO: 14 - protein, 457 aa, unidentified
MSKIVIVGANHAGTAAINTILDNYGSENEVVVFDQNSNISFLGCGMALWIGKQISGPQGLFYADKESLEAKGAKIYMESPVTAIDYDAKRVTALVNGQEHVESYEKLILATGSTPILPPIKGAAIKEGSRDFEATLKNLQFVKLYQNAEDVINKLQDKSQNLNRIAVVGAGYIGVELAEAFKRLGKEVILIDVVDTCLAGYYDQDLSEMMRQNLEDHGIELAFGETVKAIEGDGKVERIVTDKASHDVDMVILAVGFRPNTALGNAKLKTFRNGAFLVDKKQETSIPDVYAIGDCATVYDNAINDTNYIALASNALRSGIVAGHNAAGHKLESLGVQGSNGISIFGLNMVSTGLTQEKAKRFGYNPEVTAFTDFQKASFIEHDNYPVTLKIVYDKDSRLVLGAQMASKEDMSMGIHMFSLAIQEKVTIERLALLDYFFLPHFNQPYNYMTKAALKAK

SEQ ID NO: 15 - DNA, 1356 bp, Carnobacterium divergens
  source 1..1356: mol_type = genomic DNA
atgaaagtagtagtagtaggctgtacacatgcaggaacagcggcagttaagacgattttaaatgaacatccagatgcatcagtatcagtatatgagcgtaatgacaatgtctcatttctatcttgtgggattgcgttgtatgttggtggagttgtgaaagatcctgcaggtttgttttattcaagtccagaagaacttgcatcaatgggcgcgaaaattaacatggaacacaatgtgaaaaatatagataatgagaataaggtcgtagtaattgagaatttaaaaacaggcgaaacatttgaagaaagctatgataagttggtaatgacaactggatcatggccaattattcctccaattgatggaatcaatagtgaaaatattcttttgtgtaaaaactataaccaagcaaatgaaattattaaagaatcaaaaaatgctaaaaagattgtcattgttggtggtggctatattggaattgaattagttgaggcatttgcagaatctggcaagcaagtgacgctagttgatggattagatcgtattttaaacaaatatttagatgctgaattcacttctgttttagagcatgatttacaagaaagaggcgttacgctagctttaaaccaaaccgtcgagaaatttgttgccaatgaatcaggtgctgtgacagctgtgaaaacaccagttggagaatatgaggctgatttagttattttatgtgttggatttaaaccaaatactgatttgttgaaggataaagtagagatgttgccaaatggtgccatcgtagtggatgaatatatgagaacaagcgatgaagcgatttttgctgctggcgatagttgcgcggttcattataatccaactggaggctctgcgtatattccgttagctacaaatgcagttagaatgggagctttagttgggaaaaatattgtttctccaacagttaaatatcgtggcacgcaagcaacttctggtttatatttatttggttttaatataggttcaaccggattgactgaaaatagcgctcctcattttggcgtagaggttcgttcagtagttgtagaagataattatcgtccagagtttatgccgacaacagagaaagtaacgatgaaattagtttatgaagtaggaacgaatcggattgttggaggtcaaatcatgtcaaaatatgatgtgacacaatctgccaatacgttatctttatgtgttcaaaataaaatgacgattgaggatttggcttatgtagatttcttcttccaacctcactttgatcgtccttggaactatttaaatattttagcgcaagcagctgttgagcaagagcgtaaactagcaaaataa

SEQ ID NO: 16 - protein, 451 aa, Carnobacterium divergens
MKVVVVGCTHAGTAAVKTILNEHPDASVSVYERNDNVSFLSCGIALYVGGVVKDPAGLFYSSPEELASMGAKINMEHNVKNIDNENKVVVIENLKTGETFEESYDKLVMTTGSWPIIPPIDGINSENILLCKNYNQANEIIKESKNAKKIVIVGGGYIGIELVEAFAESGKQVTLVDGLDRILNKYLDAEFTSVLEHDLQERGVTLALNQTVEKFVANESGAVTAVKTPVGEYEADLVILCVGFKPNTDLLKDKVEMLPNGAIVVDEYMRTSDEAIFAAGDSCAVHYNPTGGSAYIPLATNAVRMGALVGKNIVSPTVKYRGTQATSGLYLFGFNIGSTGLTENSAPHFGVEVRSVVVEDNYRPEFMPTTEKVTMKLVYEVGTNRIVGGQIMSKYDVTQSANTLSLCVQNKMTIEDLAYVDFFFQPHFDRPWNYLNILAQAAVEQERKLAK

SEQ ID NO: 17 - DNA, 1014 bp, Geobacillus stearothermophilus
  source 1..1014: mol_type = genomic DNA
atgaaagctgcagttgtggaacaatttaaaaagccgttacaagtgaaagaagtggaaaaacctaagatctcatacggggaagtattagtgcgcatcaaagcgtgtggggtatgccatacagacttgcatgccgcacatggcgactggcctgtaaagcctaaactgcctctcattcctggccatgaaggcgtcggtgtaattgaagaagtaggtcctggggtaacacatttaaaagttggagatcgcgtaggtatcccttggctttattcggcgtgcggtcattgtgactattgcttaagcggacaagaaacattatgcgaacgtcaacaaaacgctggctattccgtcgatggtggttatgctgaatattgccgtgctgcagccgattatgtcgtaaaaattcctgataacttatcgtttgaagaagccgctccaatcttttgcgctggtgtaacaacatataaagcgctcaaagtaacaggcgcaaaaccaggtgaatgggtagccatttacggtatcggcgggcttggacatgtcgcagtccaatacgcaaaggcgatggggttaaacgtcgttgctgtcgatttaggtgatgaaaaacttgagcttgctaaacaacttggtgcagatcttgtcgtcaatccgaaacatgatgatgcagcacaatggataaaagaaaaagtgggcggtgtgcatgcgactgtcgtcacagctgtttcaaaagccgcgttcgaatcagcctacaaatccattcgtcgcggtggtgcttgcgtactcgtcggattaccgccggaagaaatacctattccaattttcgatacagtattaaatggagtaaaaattattggttctatcgttggtacgcgcaaagacttacaagaggcacttcaatttgcagcagaaggaaaagtaaaaacaattgtcgaagtgcaaccgcttgaaaacattaacgacgtattcgatcgtatgttaaaagggcaaattaacggccgcgtcgtgttaaaagtagattaa

SEQ ID NO: 18 - protein, 337 aa, Geobacillus stearothermophilus
MKAAVVEQFKKPLQVKEVEKPKISYGEVLVRIKACGVCHTDLHAAHGDWPVKPKLPLIPGHEGVGVIEEVGPGVTHLKVGDRVGIPWLYSACGHCDYCLSGQETLCERQQNAGYSVDGGYAEYCRAAADYVVKIPDNLSFEEAAPIFCAGVTTYKALKVTGAKPGEWVAIYGIGGLGHVAVQYAKAMGLNVVAVDLGDEKLELAKQLGADLVVNPKHDDAAQWIKEKVGGVHATVVTAVSKAAFESAYKSIRRGGACVLVGLPPEEIPIPIFDTVLNGVKIIGSIVGTRKDLQEALQFAAEGKVKTIVEVQPLENINDVFDRMLKGQINGRVVLKVD

SEQ ID NO: 19 - DNA, 786 bp, Priestia megaterium
  source 1..786: mol_type = genomic DNA
atgtatacagatttaaaagataaagtagttgtaattacaggtggatcaacaggtttaggacgcgcaatggctgttcgtttcggtcaagaagaagcaaaagttgttattaactattacaacaatgaagaagaagctttagatgcgaaaaaagaagtagaagaagcaggcggacaagcaatcatcgttcaaggcgacgtaacaaaagaagaagatgttgtaaaccttgttcaaacagctattaaagaattcggtacattagacgttatgattaataacgctggtgttgaaaacccagttccttctcatgagttatctttagacaactggaataaagttattgatacaaacttaacaggtgcattcttaggaagccgtgaagcaatcaaatattttgttgaaaacgacattaaaggaaacgttattaacatgtctagtgttcatgaaatgattccttggccattatttgttcattacgcagcaagtaaaggcggtatgaaactaatgacggaaacattggctcttgaatatgcgccaaaaggtatccgcgtaaataacattggaccaggtgcgatgaacacaccaattaacgcagagaaatttgcagatcctgtacaacgtgcagacgtagaaagcatgattccaatgggttacatcggtaaaccagaagaagtagcagcagttgcagcattcttagcatcatcacaagcaagctatgtaacaggtattacattatttgctgatggtggtatgacgaaatacccttctttccaagcaggaagaggctaa

SEQ ID NO: 20 - protein, 261 aa, Priestia megaterium
MYTDLKDKVVVITGGSTGLGRAMAVRFGQEEAKVVINYYNNEEEALDAKKEVEEAGGQAIIVQGDVTKEEDVVNLVQTAIKEFGTLDVMINNAGVENPVPSHELSLDNWNKVIDTNLTGAFLGSREAIKYFVENDIKGNVINMSSVHEMIPWPLFVHYAASKGGMKLMTETLALEYAPKGIRVNNIGPGAMNTPINAEKFADPVQRADVESMIPMGYIGKPEEVAAVAAFLASSQASYVTGITLFADGGMTKYPSFQAGRG